(19) United States
(12) Patent Application Publication
(10) Pub. No.: US 2026/0357217 A1
(43) Pub. Date: Nov. 12, 2026

(54) PREPARATION AND USE OF FUNCTIONAL HUMAN TISSUES

(71) Applicant: LUNDQUIST INSTITUTE FOR BIOMEDICAL INNOVATION AT HARBOR-UCLA MEDICAL CENTER, Torrance, CA (US)

(72) Inventor: Eiji YOSHIHARA, Torrance, CA (US)

(21) Appl. No.: 19/100,105

(22) PCT Filed: Aug. 2, 2023
(86) PCT No.: PCT/US2023/029297

§ 371 Date: Jan. 30, 2025

Related U.S. Application Data
(60) Provisional application No. 63/394,465, filed on Aug. 2, 2022.

(51) Int. Cl. C12N 5/071; A61K 35/39

(52) U.S. Cl. CPC C12N 5/0677; A61K 35/39; C12N 2501/115; C12N 2501/727; C12N 2506/45

(57) ABSTRACT

Compositions and methods for preparing a pancreatic islet organoid are described. The method can include dispersing stem cells in a solution comprising 0.1% (w/v) to 5% (w/v) gellan gum, dropping the solution with the dispersed stem cells to a buffer comprising Ca²⁺ to form a three-dimensional (3D) culture of stem cells, and differentiating the stem cells in the 3D culture, thereby forming a pancreatic islet organoid comprising at least 100,000 pancreatic islet cells. Also provided are methods for testing or improving the functionality of a pancreatic islet organoid.

### CROSS REFERENCE TO RELATED APPLICATIONS

This application claims the benefit under 35 U.S.C. § 119 (e) of the U.S. Provisional Application Ser. No. 63/394,465, filed Aug. 2, 2022, the content of which is hereby incorporated by reference in its entirety.

### BACKGROUND

Losing insulin producing islet β cell function or mass is the common pathological feature of diabetes. Cell replacement therapy holds promise in the treatment of diseases such as type 1 diabetes (T1D) for β cells. Human islets obtained from cadaver is precious resource to treat TID, but the shortage of islet supply limit the opportunity of this therapeutics.

Recent advances on the human pluripotent stem cell (hPSC)-derived β like cells or islet-like organoids enable to ameliorate diabetes when it's transplanted pre-clinical diabetic model mice or rodents and are expected to be an alternative resource of cadaveric islets. Recent clinical trial of hPSC-derived pancreatic progenitors (PPs), which further differentiated and maturated into insulin producing β cells and glucagon producing α cells proven the evidence this hPSC-derived cell therapy can provide safety treatment in human patient.

It is also highlighted the variation of in vivo differentiation and batch-to-batch differences of hPSC derived cell products, suggesting that limitation of scalability of high quality hPSC-derived β like cells or islet-like organoids.

### SUMMARY

The efficacy of glycemic control is dependent on the number of functional β like cells. In vitro organogenesis, however, limits the size due to lack of oxygen and nutritional circulation. In the instant disclosure, the inventors used gellan beads to encapsulate hPSCs (GB-hPSCs) and enabled them to differentiate into functional islet-like clusters, referred to herein as giant pancreatic islets (GiSLETs). The GiSLETs are about 100 times larger than human islet-like organoids (HILOs) and primary human islets.

The GiSLET technology provides fine-tuning spatial-wide transcriptome information and cell-cell communications (CCC) in human islet organogenesis and glycemic control in STZ-induced diabetic NOD-SCID mice more than 153 days in vivo. Since a greatly reduced number of clusters is required to allow equivalent glycemic control to that of human islets, this technology will provide better quality control for generated islets. In addition, it was demonstrated that GiSLETs can maintain pluripotency for more than 14 days and differentiate into not only endoderm but also ectoderm and mesoderm lineages. The GiSLET technology, therefore, has great potential to advance tissue engineering for cell therapy.

Also provided are methods for testing or improving the functionality of a pancreatic islet organoid. It is discovered herein that the FXYD Domain Containing Ion Transport Regulator 2 (FXYD2) gene is a biomarker for functional β-like cells and δ-like cells, as it is restrictedly expressed in these cells. In addition, induced FXYD2 expression can enhance glucose-stimulated insulin secretion (GSIS) in the β-like cells and δ-like cells. Mechanistically, the inventors have shown that FXYD2 physically interacts with SRC to regulate SRC-TEAD1 signaling independent with YAP signaling to modulate β cell maturity and GSIS function. FXYD2 also marked functional heterogeneity of hPSC-derived islets or GISLETs. FXYD2High HILOs showed a higher GSIS activity than FXYD2Low HILOs. These results indicate that FXYD2 marks and regulates β cell maturation via channel-sensing signal transduction and it can be used as a selection marker for functional heterogeneity of human islet organoids.

Accordingly, one embodiment of the present disclosure provides a method for preparing a tissue, preferably a pancreatic islet organoid, comprising dispersing stem cells in a solution comprising 0.1% (w/v) to 5% (w/v) gellan gum, dropping the solution with the dispersed stem cells to a buffer comprising Ca²⁺ to form a three-dimensional (3D) culture of stem cells, and differentiating the stem cells in the 3D culture, thereby forming a pancreatic islet organoid comprising at least 100,000 pancreatic islet cells.

In some embodiments, the solution comprises 0.2% (w/v) to 2% (w/v), preferably 0.5% (w/v) to 1.5% (w/v) gellan gum. In some embodiments, the solution comprises 0.2×10⁶ to 5×10⁷ stem cells, preferably 1×10⁶ to 2×10⁷ stem cells, or 1×10⁶ to 1×10⁷ stem cells.

In some embodiments, the stem cells are induced pluripotent stem cells (iPSC) or embryonic stem cells (ESC).

In some embodiments, the buffer, upon receiving the stem cells, comprises 0.05% (w/v) to 0.4% (w/v) gellan gum, preferably 0.1% (w/v) to 0.2% (w/v) gellan gum. In some embodiments, the solution and the buffer each is pre-heated to about 35-39 °C prior to be in touch with the stem cells.

In some embodiments, the method further comprises, prior to differentiation, growing the 3D culture in a growth medium. In some embodiments, the growth medium comprises a Rock inhibitor, an FGF2, or the combination thereof.

In some embodiments, the differentiation is carried out in a differentiation medium. In some embodiments, the differentiation medium comprises one or more of CHIR99021, Activin A, or KGF. In some embodiments, the pancreatic islet organoid comprises at least 200,000, 300,000, 500,000, 800,000 or 1,000,000 pancreatic islet cells. In some embodiments, the pancreatic islet organoid is at least 100 μm in diameter, or at least 200 μm, 300 μm, 400 μm, 500 μm, 600 μm, 700 μm, 800 μm, 900 μm, 1000 μm, 1200 μm, 1500 μm, 1800 μm, 2000 μm, 5000 μm, 8000 μm, or 10,000 μm in diameter.

In some embodiments, the pancreatic islet cells in the pancreatic islet organoid are capable of cell-to-cell communication. In some embodiments, the pancreatic islet cells in the pancreatic islet organoid are capable of expanding into endodermal, mesodermal and/or ectodermal layers.

In some embodiments, the method further comprises verifying the pancreatic islet organoid by detecting expression of FXYD Domain Containing Ion Transport Regulator 2 (FXYD2). In some embodiments, an expression level of FXYD2 higher than a predetermined cutoff level indicates that the pancreatic islet organoid is functional.

Also provided, in one embodiment, is a pancreatic islet organoid prepared by a method of the present disclosure. Still further provided is an in vitro prepared pancreatic islet organoid, wherein the pancreatic islet organoid is at least 100 μm in diameter, or at least 200 μm, 300 μm, 400 μm, 500 μm, 600 μm, 700 μm, 800 μm, 900 μm, 1000 μm, 1200 μm, 1500 μm, 1800 μm, or 2000 μm, 5000 μm, 8000 μm, or 10,000 in diameter, comprises at least at least 200,000, 300,000, 500,000, 800,000 or 1,000,000 pancreatic islet cells, and comprises gellan gum.

In yet another embodiment, provided is a method of providing functional pancreatic islet cells to a patient in need thereof, comprising administering to the patient one or more pancreatic islet organoid as described herein.

Also provided is a method for identifying a functional β cell or δ cell, comprising detecting the expression of the FXYD2 gene in a test cell in a biological sample, wherein expression of the FXYD2 gene identifies the test cell as a functional β cell or δ cell. In some embodiments, the biological sample comprises a pancreatic islet organoid. In some embodiments, the pancreatic islet organoid is cultured in vitro from a stem cell.

Also provided is a method for improving functionality or glucose-stimulated insulin secretion (GSIS) of a cell, comprising increasing the biological activity of the FXYD2 gene in the cell, wherein the cell is a β cell or a δ cell.

In some embodiments, the increasing of the biological activity of the FXYD2 gene comprises introducing to the cell a FXYD2 protein, or a polynucleotide encoding the FXYD2 protein. In some embodiments, the increasing of the biological activity of the FXYD2 gene comprises contacting the cell with doxycycline.

### BRIEF DESCRIPTION OF THE DRAWINGS

FIG. 1. Engineering functional giant islets in vitro. (A) Scheme of human pluripotent stem cells encapsuled gellan beads (GB-hPSCs) generation. 5 million of hPSCs (hiPSCs or hESCs) are suspended with 25 μl of 1% gellan gum. Gellan gum droplets are dropped into Ca²⁺ containing OPTI+ media and generated GB-hPSC clusters. (B) Live (green)-dead (red) cell staining in GB-hPSCs for 3 days culture in stem cell culture medium or stem cell culture medium with OPTI (OPTI+). Nucleus was stained with DAPI. Scale bar, 50 μm. % of survival (right, bar graph) was obtained from 10 independent images. (C) Scheme of transcriptome analysis. Transcriptome of day 0 (D0, pluripotent stem/PS), day 13 (D13, pancreatic progenitor/PP), day 22 (D22, immature β-like/imβ) and day 30 (D30, mature β-like/β) of differentiated HILOs and GiSLETs are analyzed. (D) Pearson correlation of whole transcriptome between samples are shown. (E) NGN3 gene expression is more enriched in day 13 of GISLETs than that of HILOs. (F) Heatmap analysis for differentially expressed genes categorized cell cycle, islet lineage specification (β cell, α cell, δ cell, γ cell, ε cell) and metabolic pathway related gene expression were shown with Z-Score. (G) GiSLETs were generated from ChiPSC12 and H1ESC. Phase-contrast image of primary human islets, HILOs (hiPSCs), GiSLETs (hiPSCs) and GiSLETs (hESCs) are shown. Insulin promoter driven GFP expressing GiSLETs (hiPSCs) are shown as green (right, bottom). Phase-contrast image of dithizone (DTZ)-stained GiSLETs (ChiPSCs) (right, upper). Scale bars, 3 mm. (H) Size distribution analysis for GiSLETs (ChiPSCs), HILOs (ChiPSCs), primary human islets. Diameter (μm) of each cluster was measured and percentage (%) of distribution was shown in Y-axis. Error bars represent ±SEM. Statistics by Student's t-test. ***p<0.001.

FIG. 2. Physiological functional insulin secretion in GiSLETs. (A, B) c-peptide secretion assay (HILO/day 30, n=3; GiSLETs-ChiPSC12/day 28, n=3; GiSLETs-H1ES/day 28, n=3; human islets, n=12). Absolute concentration of human c-peptide secretion from each single cluster was measured. G3=glucose 3 mM, G20=glucose 20 mM, SST=Somatostatin 90 nM, KCL=20 mM KCL. 0-50 μmol/L range (A) and 0-2500 μmol/L range (B) were shown. (C) Absolute human insulin secretion from human islets and GiSLETs (human islets, n=6; GiSLETs-ChiPSC12/day 38, n=6; GiSLETs-H1ES/day 38, n=6). (D) IHC for GiSLETs. INS, c-peptide, GCG, SST, CHGA, KRT19 were stained with DAPI (Nucleus staining). Scale bars, 100 μm. (E) Scheme of co-culturing system of GiSLETs (GiSLETs-ChiPSC12/day 26 and ChiPSC-derived gut organoids (HGO/day 28) and the synergistic human insulin secretion. Co-culturing of GiSLETs and HGO enhances GSIS function in GiSLETs. Error bars represent ±SEM. Statistics by Student's t-test. *p<0.05, **p<0.01, ***p<0.001.

FIG. 3. Spatial transcriptome in GISLETs. (A) Scheme of spatial transcriptomics (ST) in hislets and GiSLETs. (B) Frozen block for spatial transcriptome analyses. Area of GiSLETs and human islets (hislets) were shown in red circle. Spatial transcriptome is capable of being captured in GiSLETs (GISLET1 & GISLET2) but not in hislets due to size limitation. Scale bars, 2 mm. (C) Heatmap gene expression analysis of indicated clusters (c1-c5). Top 50 featured genes are shown. Gene Ontology (GO) analysis showed representative featured pathway in each enriched gene clusters. 5 distinguished clusters were identified. (D) Integration of scRNA-seq of human islets in spatial transcriptome. Cell type specific corresponding ST spot in hislets and GiSLETs are quantified. (E, F) Identification of spatial transcriptional features corresponding to β cells, α cells, duct cells and acinar cells in human islets (E). Unclear region-specific cell type is observed. (G, H) Identification of spatial transcriptional features corresponding to β cells, α cells, duct cells and acinar cells in GISLETs (G). Region specific cell type are observed (H). (I) Identification of 5 clusters which enriched β cell (Beta1, Beta2, Beta3), α cell (Alpha) and duct cell (Duct) transcriptional features. Beta3 locate proximally to Duct and Beta1 locate proximally to Alpha. Duct also contains acinar cell and fibroblast cell transcriptome features. (J) Cell-cell communication network among the clusters. (K) Visualization of cell-cell communication for indicated signaling include IGF, CADM, VISFATIN and LAMININ (upper panel). The network centrality scores are computed (lower panel).

FIG. 4. GiSLETs rapidly ameliorate preexisting diabetes. (A) Diabetes was induced with 180 mg/kg STZ i.p. injection in NOD-SCID mice. 2 weeks after injection, GiSLETs (c=1:1 cluster, c=2:2 clusters or c=5:5 clusters) were transplanted by kidney capsule. 500 clusters (=1,000-1,500 IEQ) of primary human islets was transplanted for positive control. Graft removal with nephrectomy (Nx) was performed indicated time point. Sacrifice for Nx or dead mice were shown (#). (B) Diabetic incidents (>250 mg/dl blood glucose) in each mouse. (C) Glucose tolerance test (GTT) with 2 g/kg glucose i.p. injection to measure blood glucose were performed at 4 weeks after transplantation. Area under the curve (AUC) was shown. (D) Glucose tolerance test (GTT) with 2 g/kg glucose i.p. injection to measure serum human c-peptide were performed at 4 weeks after transplantation. (E, F) Immunohistochemistry (IHC) for the graft of GiSLETs at 153 days after transplantation. C-peptide (c-pep), CHGA, GCG, SST, PDX-1, NKX6-1 and DAPI (nuclear staining) were performed. 2× (E) and 40× (F). Scale bars, 1 cm (E) and 100 μm (F).

FIG. 5. The geometry of computational models in simulating partial oxygen tension in hPSC clusters. The hPSC clusters are seeded into a 6-well plate with a medium height of 3 mm. (A) Top view of approximately 16,000 hPSC clusters with a diameter of 150 μm seeded into a 6-well plate with a diameter of 3.5 mm (left). Side view of a hexagonal-prism-shaped unit cell for simulating conditions (right). (B) Top view of 19 GG-hPSC clusters with a diameter of 3 mm seeded into a 6-well plate with a diameter of 3.5 mm (left). Side view of a hexagonal-prism-shaped unit cell for simulating conditions (right). (C) Oxygen simulation in hiPSC clusters (150 μm diameter) and GG-hiPSCs (3-mm diameter) cultured in static and dynamic (Rotation at 60 rpm) condition with various oxidative consumption condition (0.5, 1, 1.5 pmols/min/10⁴ cells). Oxygen tension (pO₂) are shown as heatmap and the numbers represent in the middle of each simulation shows % of hypoxic cells (pO₂<10 mmHg).

FIG. 6. Optimization of GG-hPSC cluster survival. (A-C) Optimization of GB-hPSCs culture in vitro. Encapsulation of 5 million hPSCs in 25 μl of GB generates approximately 2.06× 10⁶ cells/GG live cell cluster (A) ATP level of GB-hPSCs clusters (B) ATP levels are corresponding to the survived cell number (C) Dose dependent (1 million/1 M, 2 million/2 M and 5 million/5 M) cell survival was assessed by cellular ATP level. OPTI+ improves survival of GB-hPSCs. GG; gellan gum, MG; Matrigel. (D) Live (green)-dead (red) cell staining in GB-hPSCs for 3 days culture in stem cell culture media or stem cell culture media with OPTI (OPTI+). Nucleus was stained with DAPI. (E) Scheme of Pluripotent stem cells (PS) to Endoderm (ENDO), Mesoderm (MESO) and Ectoderm (ECTO) differentiation. (F) qPCR analyses of GG-hPSC derived ENDO, MESO, ECTO. PS marker NANOG, ENDO marker SOX17, MESO marker MSX1 and ECTO marker SOX1. (G) IHC analyses of GG-hPSC derived ENDO, MESO, ECTO. ENDO marker GATA4, MESO marker Brachyury and ECTO marker OTX2. Scale bar, 100 μm.

FIG. 7. Transcriptomics during GiSLETs differentiation. (A-C) Venn diagrams of differentially expressed genes (DEGs) in HILOs or GiSLETs compared to hPSCs in indicated post differentiated days. Down-regulated and up-regulated genes in day 13 (A), day 22 (B) and day 30 (C) were analyzed. (D-F) Gene Ontology (GO) analysis at day 13 (D), day 22 (E) and day 30 (F). Upregulated- and downregulated pathway in GiSLETs compared to HILOs at indicated post differentiated days are shown. (G) qPCR comparison of indicated gene expression among hiPSCs, HILOs (day 30), GiSLETs (day 30) and human islets. (H) Comparison of INS, NKX6-1, UCN3, IAPP, WNT4 expression in hiPSCs, HILOs and GISLETs at day 30. (I) Live cells at day 35 after differentiation to GiSLETs. Error bars represent ±SEM. Statistics by Student's t-test. (J) **p<0.01, ***p<0.001.

FIG. 8. Synchronized Ca²⁺ flux in GiSLETs. (A) Ca²⁺ excitation. (B) Representative Ca²⁺ flux measured by Incucyte SX5 live imaging. (C) Synchronized mean correlation of Ca²⁺ flux. (D) Absolute human insulin secretion from human islets and GiSLETs with indicated condition (#internal ID, d=day). Error bars represent ±SEM. Statistics by Student's t-test. *p<0.05, **p<0.01, ***p<0.001.

FIG. 9. Featured spatial transcriptome in GiSLETs. (A) H&E staining and corresponding spot overlays show the ST in GiSLETs (GISLET1 & GISLET2). 5 distinguished clusters were identified. (B) ST in GISLET1 and GISLET2. Endocrine markers (INS, GCG, SST, PPY, GHRL) are enriched in GISLET1 and exocrine (PRSS1, PRSS2, PRSS3) and ductal marker (KRT19) are enriched in GISLET2. (C) UMAP analysis of gene expression. (D) UMAP and ST of INS, CHGA, NKX6-1 and IAPP. (E) Violin plot for (D). (F) ST of INS, CHGA, CHGB, MAFB, NKX6-1 and IAPP.

FIG. 10. Featured spatial transcriptome in human islets. (A) UMAP plot for cluster-based analysis (left) and H&E staining and corresponding spot overlays show the ST in human islets (right). (B) ST of endocrine related genes in human islets. (C) ST of non-endocrine related genes in human islets. (D) Integration of scRNA-seq of human islets in spatial transcriptome. Cell type specific clusters in human islets of scRNA-seq data sets (left). UMAP plot of scRNA-seq to integrate ST clusters from GISLETs (right). Alpha, Beta1, Beta2, Delta, Duct1, Duct2, Fibro, Acini, and immune are identified in human islets.

FIG. 11. Predicted cell-cell communication network in primary human islet cells. (A) Distribution of predicted cell-cell interactions in human islets. Number of interaction and interaction weights/strength. (B) Chord diagram of significant cell-cell communication pathway in human islets. (C) Visualize cell-cell communication mediated by multiple ligand-receptors or signaling pathways in human islets.

FIG. 12. Identification of the significant cell-cell communication network in primary human islet cells. (A) Violin plot of WNT and noncanonical WNT (ncWNT) pathway related genes in human islet cell types. (B) Violin plot of VISFATIN and VEGF pathway related genes in human islet cell types. (C) The outgoing communication patterns of secreting cells, which shows the correspondence between the inferred latent patterns and cell groups, as well as signaling pathway. (D) Identify signals contributing most to outgoing signaling of indicated cell groups. (E) The incoming communication patterns of targeting cells, which shows the correspondence between the inferred latent patterns and cell groups, as well as signaling pathway. (F) Identify signals contributing most to incoming signaling of indicated cell groups.

FIG. 13. Identification of the specific significant cell-cell communication network in GiSLETs. (A) Visualization of cell-cell communication for indicated signaling include WNT, ncWNT, BMP, FGF, VEGF, COLLAGEN, DESMOSOME and MIF (upper panel). The network centrality scores are computed (lower panel). (B) Violin plot of β cell specific gene expression in Beta1 and Beta3 (Beta3 locate proximally to Duct and Beta1 locate proximally to Alpha). (C) Violin plot of YAP signaling related gene expression in Beta1 and Beta3.

FIG. 14. Histology of GiSLETs graft. (A) Scheme of transplantation of GiSLETs. GiSLETs are minced to smaller pieces prior of kidney capsules. (B) Immunostaining of frozen sectioned kidneys transplanted with GiSLETs (From Nx1 of FIG. 4A at day 16). INS, CHGA, DAPI are stained. (C) Immunostaining of frozen sectioned kidneys transplanted with GiSLETs (From Nx5 of FIG. 4A at day 153). INS, CD31, DAPI are stained. Scale bars, 100 μm.

FIG. 15. FXYD2 as novel terminal maturation marker in functional human islets. (A) Scheme of HILOs generation (left) and single hormonal insulin and glucagon cells in HILOs (right). (B) Co-immunostaining of Insulin and c-peptide in HILOs. (C) Differentially expressed genes in β, α, δ cells of human islets and HILOs. (D) Gene ontology (GO) analyses reveals enriched FXYD2 and related mineral absorption pathway in primary human β cells and δ cells compared to those of HILOs β cells and δ cells. (E) FXYD family gene expression in human islets. Single cell RNA-seq revealed enrichment of FXYD2 in primary human β cells and δ cells. (F). FXYD2 expression in mouse and human β, α, δ cells. (G) FXYD2 expression is induced during differentiation to HILOs but the expression level is ~100-folds lower than that of human β cells. (H), (I). IHC for FXYD2 reveals that FXYD2 localizes in membrane in primary human β cells (H) and human β cell lines (EndoC-BH1) (I). Error bars show s.e.m. **p<0.01.

FIG. 16. FXYD2 expression is increased during differentiation to human islet-like organoids. (A) FXYD2 expression in bulk-RNA-seq at day 0, day 13, day 22 and day 30 of differentiation from hiPSC to HILOs. (B) FXYD2 expression in qPCR at day 0, day 6, day 8, day 14, day 21, day 23 and day 25 of differentiation from hiPSC to HILOs. Error bars show s.e.m. *p<0.05, **p<0.01, *p<0.001.

FIG. 17. FXYD2 regulates GSIS and KCl stimulated insulin secretion in human β cells. (A). FXYD2 knockdown (csiFXYD2/FXYD2KI) suppress insulin secretion in EndoC-BH1 cells. (B) Dose-dependent suppression of insulin secretion by Digitoxin, measured by gaussian proinsulin Nano-Luc system in EndoC-BH1 cells. 0-10 nM digitoxin was treated for 24 hours prior of GSIS assay. (C) Dox-induced FXYD2 induction enhances insulin secretion in Endo-CBH1 cells. (D) Pharmacological FXYD2 inhibition for 24 hrs by digitoxin suppresses insulin secretion in primary human islets. Error bars show s.e.m. *p<0.05, ** p<0.01, *p<0.001.

FIG. 18. FXYD2-SRC signal complex in functional β cells. (A) pLV-V5-hFXYD2-TurboID-EGFP expression in EndoC-BH1 cells. (B) TurboID proximal labeling to identify biotinylated proximal proteins of FXYD2. Immunoprecipitation (IP) by V5-tag and immunoblot (IB) by V5 antibody (V5) or Streptavidin (SA). (C) Proteomics analyses identified unique proteins co-precipitated with V5-FXYD2. (D) IP for SA in control and V5-FXYD2-TurboID-EGFP expressed EndoC-BH1 cells. IB for V5, SRC, CTTN, ATP1A1 and SA. (E) WB analysis for SRC signaling in dox-induced FXYD2 overexpression (dox +) in EndoC-BH1 cells. (F) Quantification of WB results (E). (G) human insulin secretion assay in primary human islets. SRC activator (SRCa) or SRC inhibitor (SRCi) were pretreated 48 hours prior of GSIS assay. Propafenone (Referred to P2E2) was acutely treated during GSIS assay. SRCi suppressed Propafenone augmented GSIS in primary human islets. (H) Working model of FXYD2-SRC signal pathway.

FIG. 19. Sodium Ci enhances insulin secretion in EndoC-BH1 cells and primary human islets. (A) Co-IP, V5-FXYD2 and endogenous SRC. (B) Small molecule screening on insulin NanoLuc system in EndoC-BH1 cells. (C) Sodium Cis stimulate insulins secretion in primary human islets (right). (D) Sodium Cis enhances SRC Thr416 phosphorylation. (E) Quantification of WB for pSRC (Y416), non-pSRC (Y416) and β actin. Error bars show s.e.m. *p<0.05, **p<0.01, *p<0.001.

FIG. 20. FXYD2 overexpression and pharmacological inhibition inversely regulates transcriptome in human β cells. (A). Dynamic changes of gene expression by dox-inducible FXYD2 overexpression (dFXYD2OE) in EndoC-BH1 cells. (B). Gene ontology (GO) analysis of upregulated (Up) or downregulated (Down) regulated genes by dFXYD2OE. (C). dFXYD2OE enhances the gene necessary for functional β cells, while down-regulate cell cycle related genes. Heatmap (Z-Score). (D). Reciprocal metabolic gene regulation by dFXYD2OE and pharmacological FXYD2 inhibition (FXYD2i). Heatmap (Z-Score). (E) Motif analyses revealed that the promoter region of gene clusters regulated by FXYD2 commonly shows transcription factors (TFs) binding sites listed above. (F) dFXYD2OE or dFXYD2KD-HILOs at day 27 were treated with dox for 4 days and gene expression was analyzed.

FIG. 21. FXYD2 overexpression enhances maturity related gene expression and insulin secretion in EndoC-BH1 cells. (A). Venn Diagram of upregulated genes by FXYD2OE and downregulated genes by FXYD2i. (B). Downregulated pathway by FXYD2i and/or Upregulated pathway by dFXYD2OE. Number in the heatmap shows the gene number and color show the p value. (C) Experimental scheme of constitutive FXYD2 overexpression (cFXYD2OE) in EndoC-BH1 cells. (D) qPCR analyses for INS and MAFA in control and cFXYD2OE EndoC-BH1 cells. (E) GSIS assay of control and cFXYD2OE EndoC-BH1 cells. (F) Quantification of protein expression in propafenone treated, cFXYD2OE and propafenone+cFXYD2OE EndoC-BH1 cells. Error bars show s.e.m. *p<0.05, **p<0.01, *p<0.001.

FIG. 22. TEAD1 regulates maturity related gene expression in EndoC-BH1 cells. (A) WB analyses of propafenone treatment with or without V5-tagged cFXYD2OE in EndoC-BH1 cells. Quantification of WB for TEAD1 and pSRC (Thr416). (B) Relative gene expression for TEAD1, MAFA, UCN3 and IAPP in dox-induced TEAD1OE and TEAD1KD in EndoC-BH1 cells. (C) Model of the FXYD2-SRC-TEAD1.

FIG. 23. FXYD2 is a functional marker of hPSC-derived islets. (A) Drug screening in FXYD2 promoter driven Luciferase and mCherry expressing EndoC-BH1 cells identified Dex and T3 upregulates FXYD2 promoter activity. (B) Dex and T3 synergistically enhances FXYD2 gene expression in EndoC-BH1 cells. (C) Osmotic pressure by NaCl stimulation enhances FXYD2 and ATP1B1 expression in EndoC-BH1 cells and hiPSC-derived β like cells. (D) Dex and NaCl synergistically stimulate FXYD2 expression in EndoC-BH1 cells. (E) Cellular ATP level (ratio). FXYD2OE shows resistance to NaCl (Osmitic pressure) induced cell death in EndoC-BH1 cells. In contrast, FXYD2KD shows enhanced sensitivity to NaCl (Osmitic pressure) induced cell death in EndoC-BH1 cells. (F) FXYD2^{High}HILOs can be identified by FXYD2-fLuc-mCherry expression. FXYD2 enriched human β cells were isolated from HILOs. (G) Size matched pseudo HILOs from FXYD2 low expressing (FXYD2^{Low}pHILOs) and FXYD2 high expressing HILOs (FXYD2^{High}pHILOs). (H) GSIS function in FXYD2^{Low}pHILOs and FXYD2^{High}pHILOs. Error bars show s.e.m. *p<0.05, **p<0.01, *p<0.001.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### DEFINITIONS

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, “stem cell” defines a cell with the ability to divide for indefinite periods in culture and give rise to specialized cells. Non-limiting examples of types of stem cells include somatic (adult) stem cells, embryonic stem cells, parthenogenetic stem cells and/or induced pluripotent stem cells (iPS cells or iPSCs).

As used herein, the term “pluripotent stem cells” refers to cells that are: (i) capable of indefinite proliferation in vitro in an undifferentiated state; (ii) maintain a normal karyotype through prolonged culture; and (iii) maintain the potential to differentiate to derivatives of all three embryonic germ layers (endoderm, mesoderm, and ectoderm) even after prolonged culture. Non-limiting examples of currently available pluripotent stem cells include embryonic stem cells and iPSCs.

The term “organoid” refers to an in vitro generated body that mimics organ structure and function. “Organoid” and “mini organ” are used interchangeably herein. A “pancreatic islet organoid” is an in vitro generated cell cluster that mimics structure and function of a pancreatic islet. Exemplary functions of a pancreatic islet include, without limitation, glucose-stimulated insulin secretion (GSIS), potassium chloride (KCl)-stimulated insulin secretion, GLP-1 stimulated insulin secretion, somatostatin secretion, or glucagon secretion. “Pancreatic islet organoid” and “mini pancreatic islet” are used interchangeably herein. A “pancreatic organoid” is an in vitro generated body that mimics structure and function of a pancreas. Exemplary functions of a pancreas include, without limitation, endocrine secretion of hormones, such as glucose and glucagon, that regulate glucose metabolism and blood glucose concentration, and exocrine secretion of digestive enzymes that help break down carbohydrates, proteins, and lipids. “Pancreatic organoid” and “mini pancreas” are used interchangeably herein.

The present disclosure provides compositions and methods that are useful for generating scalable, functional, vascularized organoids in vitro, particularly human pancreatic or pancreatic islet organoids.

Also provided are methods for using the islets for therapeutic purposes. Islet transplantation is a therapy for treating insulin deficient diabetes such as type 1 and late stage type 2 diabetes. Thus, in another aspect, the present disclosure provides methods of treating a pancreatic disease such as type 1 or type 2 diabetes comprising administering a pancreatic or pancreatic islet organoid of the disclosure to a subject (e.g., a mammal such as a human) by transplantation. One embodiment is a method of treating a subject suffering from or susceptible to a pancreatic disease (e.g., type 1 diabetes) or disorder or symptom thereof. The method includes the step of transplanting a pancreatic or pancreatic islet organoid of the disclosure to the mammal sufficient to treat the disease or disorder or symptom thereof, under conditions such that the disease or disorder is treated.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

### Preparation of Functional Tissues

Experimental data in the accompanying examples demonstrated the engineering of giant pancreatic islets from hPSCs as a new way of mass production and mimetics of functional pancreatic islet organogenesis. Mass production of functional human islets from hPSCs can be challenging due to the difficulty of handling small clusters over 30 day periods of differentiation.

The new giant pancreatic islets (GiSLETs) technology circumvented the limitation of islet size using galen beads (GB) techniques. The advantage of GiSLETs, compared to classical stem cell-derived islets, include: 1) generation of larger amount of functional insulin-producing cells in each cluster; 2) production of approximately 100 times larger volume of insulin by GiSLETs; 3) less chance of cluster loss during differentiation; 4) fine-mapping spatial transcriptome; 5) easier handling for co-culture with other tissues (such as gut organoids); 6) in vitro long-term culture for approximately 279 days; and 7) significant reduction of the cost of differentiation. The instant inventors were able to generate 3-40 clusters of GB-hPSCs in 1 well with 2 ml medium in 6 wells having 7.5×10⁷-1.0×10⁸ hPSCs. These eventually generated 4×10⁷-8×10⁷ cells, which was approximately 10 times higher yield than with the currently available methods for hPSC β-like cells generation (approximately 5×10⁶ cells in 1 well of 6 well), indicating that cost-effective generation of functional insulin-producing cells for cell therapy in diabetes can be achieved by GiSLET technology.

Hyaluronic acid-based hydrogel (HA) or matrigel (MG) is often used for the scaffold of hPSCs or hPSC-derived organoids and for related cell manufacturing applications. However, HA and MG have limited potential in large-volume of cell production due to the complex gel composition and high cost. The newly developed GG-based GiSLETs provide a cost-effective way to produce significantly larger amounts of functional β-like cells for cell therapy in diabetes.

Advanced differentiation and maturation marker expression in GiSLETs was observed as compared to that in HILOs on the same day of differentiation. This finding appeared to parallel the elevation of NGN3 gene expression. The F-actin-YAP1-Notch mechano-signaling axis had previously been identified to repress NGN3 transcription in PP-EP stage. Similarly, cytoskeletal depolymerization by small molecules enhanced NGN3 expression in PP-EP stage. Also observed was inferred CCC identified region specific influence of YAP signaling pathway in GiSLETs. The results demonstrate that region-specific CCC can contribute to human pancreas differentiation and maturation. High-O₂ condition is another key determining factor of early-stage of pancreatic differentiation that enhances NGN3 expression by suppressing hypoxia-inducible factor-1α (HIF1α) in both mouse embryo and human ESCs. Our O₂ simulation study found that GG encapsulation using these conditions (with dynamic rotation) has higher oxygen supply compared to that without GG. This could partly explain why the GiSLETs showed enhanced lineage markers throughout the course of differentiation compared to HILOs.

Understanding of spatiotemporal gene regulation during organogenesis can provide essential knowledge of how functional organ generation can be achieved. Single cell transcriptomes unveiled essential pathways for human pancreatic islet development. Combining ST analyses with scRNA-seq can further deepen spatiotemporal information; however, the current commercially available genome-wide ST technologies is not applicable to smaller organs, such as pancreatic islets due to the resolution limitation (approximately 55 μm). It is herein found that GiSLETs (approximately 3,000 μm) showed clearer distinction of region-specific cell types than human islets (approximately 150 μm). The result indicated that GB-hPSCs differentiated to one giant islet rather than a few hundreds of small islet clusters and neighboring cells influenced the fate-determination during human islet organogenesis. Despite the limited resolution of ST, our data showed that the region specific CCC can be captured in GiSLETs.

Clinical trials with hPSC-derived PP cells reported the safety of this therapeutic in patients with T1D. Although the safety and proof of insulin producing cell survival in patients with TID was provided, PP-derived cells were found to not improve glycemia in patients with T1D due to differentiation of majority of PP-derived cells into glucagon producing a-like cells, that caused shortage of production of functional insulin producing cells. This highlighted the technical difficulty of controlling the lineage fate of PP cells in vivo, and indicated that more matured stem cell derived islets for therapeutically functional cell therapy in diabetes are required. Recent development has enabled the designer cells, which were engineered to express insulin and L-type voltage gated channel to secrete insulin via electric devices, which can be further support the functionality of the cells.

Taken together, the accompanying experimental examples demonstrated that the engineering encapsulated hPSCs, which can be cultured in vitro, is a cost-effective method to generate functional insulin producing cells. The approach can open new opportunities to design, and add-on to practical mass production of desired cells for studying spatiotemporal signaling and organ generation, and practical scaling of cell therapy in human diseases.

In accordance with one embodiment of the present disclosure, therefore, provided is a method for preparing a tissue, such as a pancreatic islet organoid. In some embodiments, the method entails dispersing stem cells in a solution that contains gellan gum, dropping the solution with the dispersed stem cells to a buffer to form a three-dimensional (3D) culture of stem cells. The buffer preferably contains Ca²⁺. The 3D culture can then be differentiated to form a pancreatic islet organoid.

The term “gellan gum” (GG) refers to a polysaccharide having a straight chain with a repeating unit that has any one of the following molecular structures:

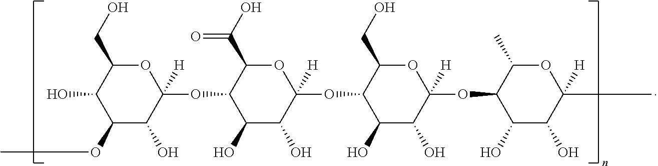

The repeating unit of the polymer is a tetrasaccharide, which consists of two residues of D-glucose and one of each residues of L-rhamnose and D-glucuronic acid. The tetrasaccharide repeat has the following structure:

### [D-Glc(β1→4)D-GlcA(β1→4)D-Glc(β1→4)L-Rha(α1→3)],

Gellan gum products are generally put into two categories, low acyl and high acyl depending on number of acetate groups attached to the polymer. The low acyl gellan gum products form firm, non-elastic, brittle gels, whereas the high acyl gellan gum forms soft and elastic gels.

In some embodiments, the gellan gum has a molecular weight of 1×10⁵ Da to 20×10⁵ Da. In some embodiments, the gellan gum has a molecular weight that is at least 0.01×10⁵ Da, 0.02×10⁵ Da, 0.05×10⁵ Da, 0.1×10⁵ Da, 0.1×10⁵ Da, 0.2×10⁵ Da, 0.5×10⁵ Da, 1×10⁵ Da, 2×10⁵ Da, 3×10⁵ Da, 4×10⁵ Da, or 5×10⁵ Da. In some embodiments, the gellan gum has a molecular weight that is not greater than 100×10⁵ Da, 50×10⁵ Da, 20×10⁵ Da, 10×10⁵ Da, 8×10⁵ Da, 6×10⁵ Da, 5×10⁵ Da, 4×10⁵ Da, 3×10⁵ Da, 2×10⁵ Da, or 1×10⁵ Da.

In some embodiments, the solution contains 0.1% (w/v) to 5% (w/v) gellan gum. In some embodiments, the solution contains 0.2% (w/v) to 2% (w/v) gellan gum. In some embodiments, the solution contains 0.5% (w/v) to 1.5% (w/v) gellan gum. In some embodiments, the gellan gum concentration is at least 0.01%, 0.02%, 0.05%, 0.1%, 0.2%, 0.5%, 0.7%, 1%, 1.2%, 1.5%, 2%, 2.5%, 3%, 3.5% or 4% (w/v). In some embodiments, the gellan gum concentration is not higher than 10%, 9%, 8%, 7%, 6%, 5%, 4%, 3.5%, 3%, 2.5%, 2%, 1.5%, 1.2%, 1%, 0.8%, 0.5%, 0.2% or 0.1% (w/v).

In some embodiments, the solution includes 0.2×10⁶ to 5×10⁷ stem cells, preferably 1×10⁶ to 2×10⁷ stem cells, or 1×10⁶ to 1×10⁷ stem cells. In some embodiments, the stem cells include induced pluripotent stem cells (iPSC). In some embodiments, the stem cells include embryonic stem cells (ESC).

Once the stem cell/gellan gum solution is dispersed in the buffer, the concentrations of the stem cells and the gellan gum will be reduced. In some embodiments, the dispersed solution, which forms the 3D culture, includes 0.05% (w/v) to 0.4% (w/v) gellan gum. In some embodiments, the 3D culture includes 0.1% (w/v) to 0.2% (w/v) gellan gum. In some embodiments, the 3D culture includes at least 0.001%, 0.005%, 0.01%, 0.02%, 0.05%, 0.1%, or 0.2% (w/v) gellan gum. In some embodiments, the 3D culture includes no more than 2%, 1%, 0.5%, 0.2%, 0.15%, 0.1%, or 0.05% (w/v) gellan gum.

In some embodiments, the solution and the buffer each is pre-heated to about 35-39 °C prior to be in touch with the stem cells.

In some embodiments, prior to differentiation, the 3D culture is grown in a growth medium. The growth medium may be based on commonly used cell culture media. In some embodiments, the growth medium includes a Rock inhibitor. In some embodiments, the growth medium includes an FGF2.

The term “ROCK inhibitor” means a substance inhibiting Rho kinase (ROCK: Rho-associated, coiled-coil containing protein kinase) and may be substance inhibiting any of ROCK I and ROCK II. The ROCK inhibitor is not particularly limited as long as the ROCK inhibitor has the function described above. Examples of the ROCK inhibitor that can be used include: N-(4-pyridinyl)-4β-[(R)-1-aminoethyl]cyclohexane-1α-carboxamide (Y-27632), fasudil (HA1077), (2S)-2-methyl-1-[(4-methyl-5-isoquinolinyl]sulfonyl]hexahydro-1-H-1,4-diazepine (H-1152), 4β-[(1R)-1-aminoethyl]-N-(4-pyridyl)benzenecarboxamide (Wf-536), N-(1H-pyrrolo[2,3-b]pyridin-4-yl)-4PER (R)-1-aminoethyl]cyclohexane-carbox-amide (Y-30141), N-(3-{[2-(4-amino-1,2,5-oxadiazol-3-yl)-1-ethyl-1H-imidazo[4,5-c]pyridin-6-yl]oxy}phenyl)-4-{[2-(4-morpholinyl)ethyl]-oxy}benzamide (GSK269962A) and N-(6-fluoro-1H-indazol-5-yl)-6-methyl-2-oxo-4-[4-(trifluoromethyl)phenyl]-3,4-dihydro-1H-pyridine-5-carboxamide (GSK429286A); antibodies (including functional fragments), antisense nucleic acids, and siRNA against ROCK; ROCK antagonists and dominant negative forms; and other ROCK inhibitors known in the art.

In some embodiments, the differentiation is carried out in a differentiation medium. In some embodiments, the differentiation medium includes one or more of CHIR99021, Activin A, or KGF.

The prepared pancreatic islet organoid, in some embodiments, includes at least 10,000 pancreatic islet cells. In some embodiments, the prepared pancreatic islet organoid includes at least 50,000, 100,000, 200,000, 300,000, 500,000, 800,000 or 1,000,000 pancreatic islet cells.

In some embodiments, the pancreatic islet organoid is at least 100 μm in diameter, or at least 200 μm, 300 μm, 400 μm, 500 μm, 600 μm, 700 μm, 800 μm, 900 μm, 1000 μm, 1200 μm, 1500 μm, 1800 μm, 2000 μm, 5000 μm, 8000 μm, or 10,000 μm in diameter.

In some embodiments, the pancreatic islet cells in the pancreatic islet organoid are capable of cell-to-cell communication. In some embodiments, the pancreatic islet cells in the pancreatic islet organoid are capable of expanding into endodermal, mesodermal and/or ectodermal layers. In some embodiments, the pancreatic islet cells in the pancreatic islet organoid are capable of expanding into all of endodermal, mesodermal and ectodermal layers.

The functionality of the pancreatic islet cells in the pancreatic islet organoid can be verified with suitable biomarkers. It is discovered herein that the expression of FXYD Domain Containing Ion Transport Regulator 2 (FXYD2) is restricted to functional β cells and δ cells. Accordingly, if the cells in the pancreatic islet organoid are detected to express FXYD2, the cells can be confirmed as functional pancreatic islet cells. In some embodiments, a positive expression is an expression level higher than a predetermined cutoff level which can be empirically determined.

Also provided, in one embodiment, is a pancreatic islet organoid prepared by a method of the present disclosure. Still further provided is an in vitro prepared pancreatic islet organoid, wherein the pancreatic islet organoid is at least 100 μm in diameter, or at least 200 μm, 300 μm, 400 μm, 500 μm, 600 μm, 700 μm, 800 μm, 900 μm, 1000 μm, 1200 μm, 1500 μm, 1800 μm, or 2000 μm, 5000 μm, 8000 μm, or 10,000 in diameter, comprises at least at least 200,000, 300,000, 500,000, 800,000 or 1,000,000 pancreatic islet cells, and includes gellan gum.

In yet another embodiment, provided is a method of providing functional pancreatic islet cells to a patient in need thereof, comprising administering to the patient one or more pancreatic islet organoid as described herein.

The therapeutic methods of the disclosure (which include prophylactic treatment) in general comprise administration (in particular, transplantation) of an effective amount of a pancreatic or pancreatic islet organoid to a subject (e.g., animal, human) in need thereof, including a mammal, particularly a human. The administration of the pancreatic or pancreatic islet organoid may be by any suitable means that results in an amount of the organoid that, combined with other components, is effective in ameliorating, reducing, or stabilizing a pancreatic disease such as type 1 or type 2 diabetes.

In some aspects, the subject is further administered an immunosuppressant. The immunosuppressant can be administered to the subject before, during, or after the subject is administered (e.g., transplanted) with the organoid. The immunosuppressive agent can be an agent that inhibits or prevents rejection (e.g., acute rejection) of the transplanted organoid upon transplantation, or an agent that maintains immunosuppression after the transplantation. Immunosuppressants include, but are not limited to, basilizimab, antithymocyte globulin, alemtuzumab, prednisone, azathioprine, mycophenolate, cyclosporine, sirolimus, and tacrolimus.

In some embodiments, islets of the subject are removed prior to transplanting the organoids of the disclosure. In some other embodiments, pancreatic islet organoids are transplanted into a subject by injection into the upper abdomen of the subjects. In some embodiments, the pancreatic islet organoids are injected into the liver. The pancreatic islet organoids can be injected into the subject using a catheter. In some other embodiments, the pancreatic organoid or pancreatic islet organoid is administered to the subject by surgery. In another embodiment, pancreatic islet organoids are transplanted onto the omentum. For omentum transplantation, a layering technique can be used in which the islet organoid (or cells thereof) are combined with autologous plasma and are laparoscopically layered onto the omentum. A solution (20 ml) containing recombinant thrombin (1000 U/ml) is next layered over the islet organoid, followed by another layer of autologous plasma to produce a biodegradable biologic scaffold that can survive and function in the patient for at least a year (See, e.g., Baidal, D. et al., 2017, N. Engl. J. Med., 376:19). In another embodiment, hydrogel biomaterials that mitigate an immune response by the recipient can be used for islet organoid transplantation. (See, e.g., Vegas, A. et al., 2016, Nature Biotechnology, 34:345-352).

To further reduce an immune reaction to the transplanted organoid in the subject, the organoid can be encapsulated in a hydrogel and then transplanted in the subject. Such methods of transplantation are further described in Vegas et al., Nature Medicine 2016, doi: 10.1038/nm.4030; Vegas et al., Nature Biotechnology 2016, doi: 10.1038/nbt.3462. In some embodiments, the hydrogel contains an alginate or alginate derivative (e.g., triazole-thiomorpholine dioxide). Various modifications of alginate hydrogels that substantially reduce inflammatory or fibrotic effects of alginate hydrogels have also been identified (Vegas et al., Nature Biotechnology 2016, doi: 10.1038/nbt.3462). Thus, in some other embodiments, the hydrogel contains a chemical modification that reduces an inflammatory effect of the transplanted organoid in the subject.

Pancreatic islet organoids and pancreatic organoids of the disclosure can be useful for modeling diseases of the pancreas in vitro or in vivo. Such pancreas disease models can be used to identify drugs that are useful for treatment of a pancreatic disease. Thus, in some aspects, the disclosure provides methods for identifying modulators, i.e., candidate or test compounds or agents (e.g., proteins, peptides, peptidomimetics, peptoids, polynucleotides, small molecules or other drugs) that are useful for the treatment of a pancreatic disease, particularly type 2 diabetes and/or pancreatic cancer. In one embodiment, the agent modulates an activity of an organoid of the disclosure.

### Methods for Identifying Functional Pancreatic Islet Cells

As provided, it is discovered herein that the expression of FXYD Domain Containing Ion Transport Regulator 2 (FXYD2) is restricted to functional β cells and δ cells, including primary β cells and δ cells and stem cell-derived human β-like cells and δ-like cells. Accordingly, if the cells in the pancreatic islet organoid are detected to express FXYD2, the cells can be confirmed as functional pancreatic islet cells. In some embodiments, a positive expression is an expression level higher than a predetermined cutoff level which can be empirically determined.

Accordingly, one embodiment of the present disclosure provides a method for identifying a functional pancreatic islet cell. The method, in some embodiments, entails detecting the expression of the FXYD2 gene in a test cell in a biological sample, wherein expression of the FXYD2 gene identifies the test cell as a functional pancreatic islet cell. In some embodiments, the functional pancreatic islet cell is a β cell or δ cell, or a functional β-like cell or δ-like cell.

In some embodiments, the biological sample comprises a pancreatic islet organoid. In some embodiments, the pancreatic islet organoid is cultured in vitro from a stem cell.

In some embodiments, the expression of the FXYD2 gene is mRNA expression. In some embodiments, the expression of the FXYD2 gene is protein expression. In some embodiments, the detection is carried out with a technology known in the art, such as with a nucleic acid probe or with an anti-FXYD2 antibody, without limitation.

### Methods for Improving the Functionality of Pancreatic Islet Cells

It is further discovered herein that induction of expression of the FXYD2 gene in a pancreatic islet cell can improve its functionality. For instance, when the expression of FXYD2 was induced by doxycycline, the glucose-stimulated insulin secretion (GSIS) of the cells was increased significantly.

Accordingly, the present disclosure provides a method for improving the functionality or glucose-stimulated insulin secretion (GSIS) of a pancreatic islet cell by increasing the biological activity of the FXYD2 gene in the cell. In some embodiments, the pancreatic islet cell is a β cell or a δ cell.

In some embodiments, increase of the biological activity of the FXYD2 gene is by increasing the expression of the gene. In some embodiments, increase of the biological activity of the FXYD2 gene is by increasing the activity of the protein.

There are an abundance of techniques known in the art to increase the expression or activity of a gene. In one aspect, the gene level is increased by increasing the amount of a polynucleotide encoding gene, as provided above, wherein that polynucleotide is expressed such that new gene is produced. In another aspect, increasing the gene level is accomplished by increasing the transcription of a polynucleotide encoding gene, or alternatively translation of gene, or alternatively post-translational modification, activation or appropriate folding of gene. In yet another aspect, increasing gene level is increased by increasing the binding of the protein to appropriate cofactor, receptor, activator, ligand, or any molecule that is involved in the protein's biological functioning. In some embodiments, increasing the binding of gene to the appropriate molecule is increasing the amount of the molecule. In one aspect of the embodiments, the molecule is the gene protein. In another aspect of the embodiments, the molecule is a small molecule. In a further aspect of the embodiments, the molecule is a polynucleotide.

Methods of increasing the amount of polynucleotide in a cell are known in the art and can be modified for increasing the amount of a polynucleotide encoding gene. In one aspect, the polynucleotide can be introduced to the cell and expressed by a gene delivery vehicle that can include a suitable expression vector.

Suitable expression vectors are well-known in the art, and include vectors capable of expressing a polynucleotide operatively linked to a regulatory element, such as a promoter region and/or an enhancer that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

Non-viral vector may include plasmid that comprises a heterologous polynucleotide capable of being delivered to a target cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject. The term vector may include expression vector and cloning vector.

Chemical or pharmaceutical agents are also known in the art that can increase the expression of the FXYD2 gene. In one embodiment, the agent is doxycycline ((4S,4aR,5S,5aR,6R,12aS)-4-(Dimethylamino)-3,5,10,12,12a-pentahydroxy-6-methyl-1,11-dioxo-1,4,4a,5,5a,6,11,12a-octahydrotetracene-2-carboxamide).

In some embodiments, the method is carried out in vitro. In some embodiments, the method is carried out in vivo. In some embodiments, the method is carried out ex vivo. For in vivo applications, the pancreatic islet cell may be a cell in a patient suffering from a disease associated with dysfunctional pancreatic islet cells, such as diabetes.

### EXAMPLES

### Example 1. Engineering Functional Human Giant Islets In Vitro

This example demonstrates that functional giant islet technology can overcome the existing limitations of in-vitro organogenesis. Gellan bead (GB)-coated human pluripotent stem cells (hPSCs) clusters were found to differentiate into pancreatic lineage, generating giant pancreatic islets (GiSLETs). The GiSLETs were approximately ~100 times larger than human islets or human islet-like organoids (HILOs), and showed functional insulin secretion. Spatial transcriptome analysis revealed region specific human islet like signatures and the unique cell-cell communications (CCC). Five GiSLETs were sufficient to ameliorate hyperglycemia in diabetic NOD-SCID mice for more than 150 days. Taken together, the instant GiSLET technology can overcome the current roadblock in the mass production of hPSC-derived functional human islets and provide a unique model for studying spatiotemporal regulation of human pancreas organogenesis and CCC.

Functional human organoids recapture the human organogenesis and physiological responses. However current technology limits the scalability and size of the organoids due to shortage of nutrition, accumulation of waste and hypoxia inside of organoids. Here we show that the functional giant organoids technology overcome the current limitation of in vitro organogenesis. Gellan bead (GB)-coated human pluripotent stem cells (hPSCs) clusters can grow and differentiated into endoderm, mesoderm and ectoderm lineage. The human giant islets (GiSLETs) can grow ~100 times bigger than primary human islets or human islet-like organoids (HILOs) from pluripotent stem cells (PSCs). The GiSLETs showed functional glucose-stimulated insulin secretion in vitro and in vivo. They also showed improved efficacy of pancreatic fate determination than HILOs due to expansion of NGN3 positive endocrine progenitors. Spatial transcriptome analysis revealed that region specific human islet like signatures and heterogeneity of the clusters are capable to monitor in GISLETs. Five of GiSLETs were sufficient to ameliorate hyperglycemia in streptozotocin (STZ) induced diabetic NOD-SCID mice more than 150 days. GiSLETs enhanced GSIS activity through organ-organ interaction with human intestinal organoids. The GiSLETs technology, therefore, can overcome the current road-block of mass production of functional human islets from PSCs and provide a unique opportunity for studying spatiotemporal regulation of human organogenesis and organ-organ communications in vitro.

### Results

### Engineering of Functional Giant Pancreatic Islet

The challenge of tissue engineering is characterized by the difficulty of circulation of nutrients and oxygen when the target tissue's size exceeds a few hundred mm in diameter, since the in vitro engineered organoids or tissues lack a functional vasculature component, which is necessary for nutrient and oxygen supply to the inner core of the cells in vivo. We reasoned that enhancing the perfusion of nutrients and oxygen to the center of cell clusters may enable us to create functional giant cell clusters.

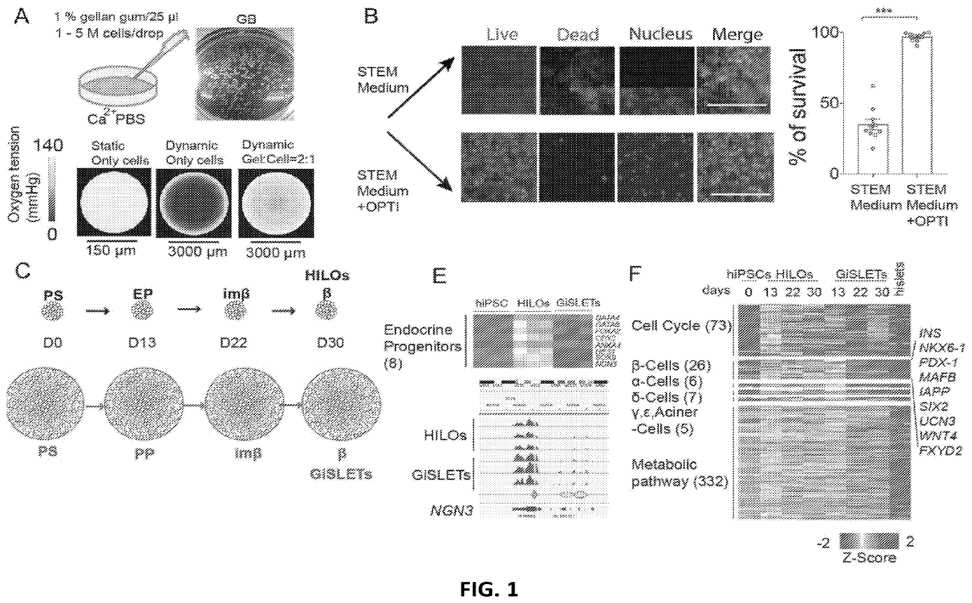

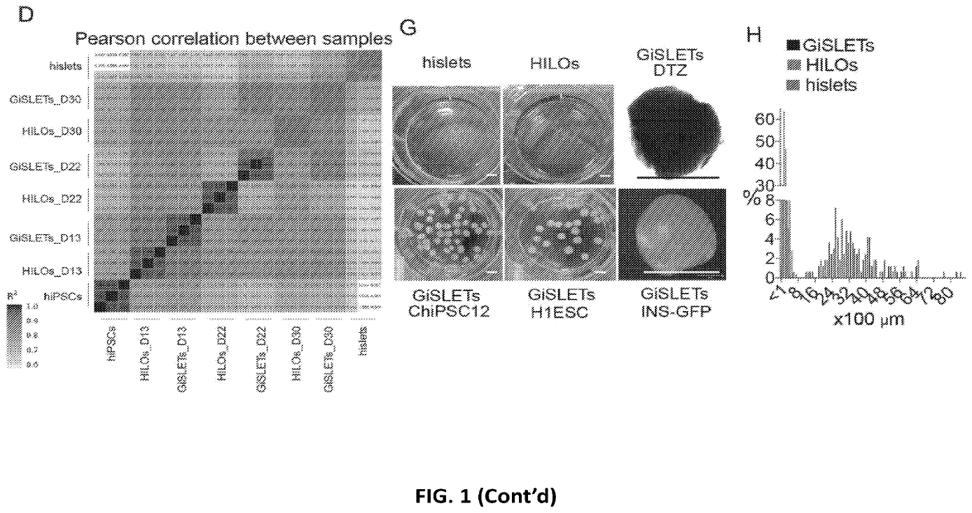

Gellan-gum (GG) hydrogel is a cost-effective natural biodegradable scaffold, which is used for tissue engineering. We have demonstrated that low-concentration GG (0.015%) in 3D culture system can enable the generation of functional human islet-like organoids. Using the natural property of GG, we generated 1% GG-containing GB-hPSC clusters in Ca²⁺-containing buffer (FIG. 1A top). We found that basal oxygen consumption rate (OCR) is increased during the course of differentiation from hPSC to insulin producing cells, therefore, we applied various basal OCR from 0.5 μmol/min/10⁴ (hPSCs) to 1.5 μmol/min/10⁴ (hPSC-derived β cell-like cells) for our oxygen simulation study.

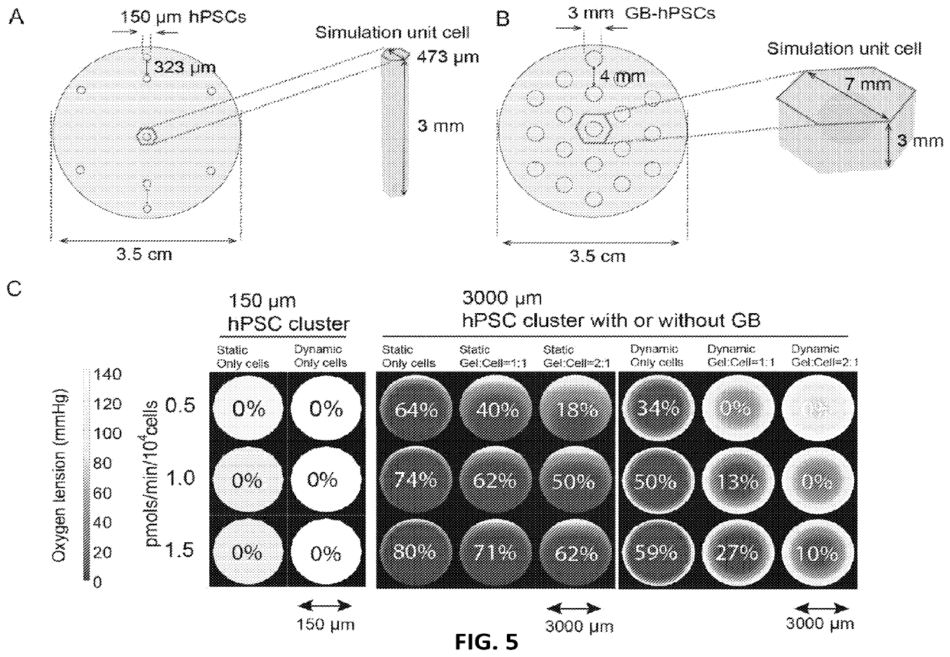

Our computational simulation study identified that GG encapsulation enhances partial oxygen tension, theoretically enabling us to avoid hypoxia (<10 mmHg) (FIG. 1A bottom, FIG. 5A, B). In static culture condition, 64%-80% of the cells, in a 3-mm hPSC cluster with 100% cell component, was exposed to the severe hypoxia of <10 mmHg, whereas 18%-62% of the cells in a 3-mm hPSC cluster, with 2:1 gel-cell component, was exposed to the severe hypoxia, demonstrating reduced hypoxia in the non-O₂-consuming gel. Furthermore, in the improved oxygen condition of the medium, in the shaker, 0%-10% of the cells in a 3-mm GB-hPSC cluster with 100% cell component were exposed to severe hypoxia of <10 mmHg, and 10% of the cells in a 3-mm hPSC cluster with 2:1 gel-cell component was exposed to severe hypoxia, which demonstrated the synergistic effects of mixing gel and shaker condition on reduced hypoxia in the large clusters (FIG. 5C).

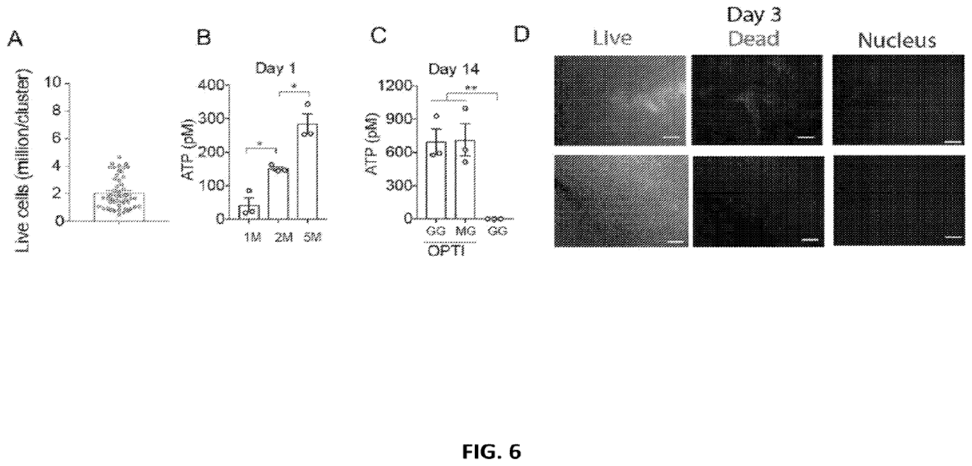

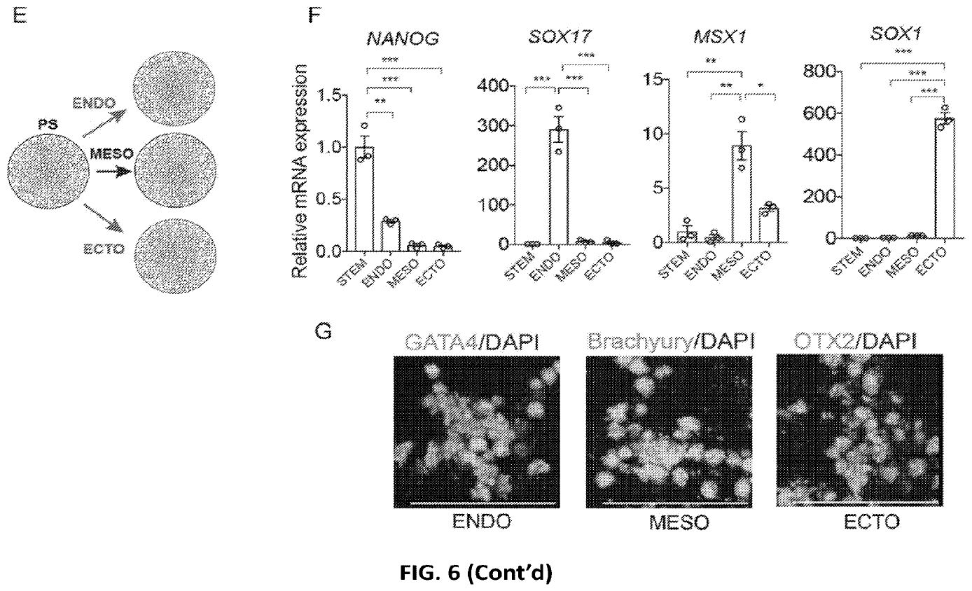

We encapsulated 5×10⁶ cells/25 ml of 1% GG. This provided GG:hPSCs=1-2:1 ratio and contained approximately 2.06×10⁶ cells in 25 ml of GB-hPSC clusters (FIG. 6A). We found GB-hiPSCs in stem cell culture medium to rapidly induced cell death within 3 days of encapsulation, whereas additional Rho kinase inhibitor (ROCKi) and fibroblast growth factors-2 (FGF2) treatment (OPTI) improved hPSC survival from 35% to 96% in GG (FIG. 1B, FIG. 6B-D). We confirmed that the ATP level, which indicates the cellular survival, increased depending on the number of encapsulated cells (FIG. 6B). We found that GB-hPSCs, similar to matrigel (MG)-embedded hiPSCs, can survive in commercial stem cell culture medium with OPTI for more than 14 days (FIG. 6C). The results, therefore, demonstrate that GB-hPSCs are cost-effective scalable tools for creating giant hPSC-clusters.

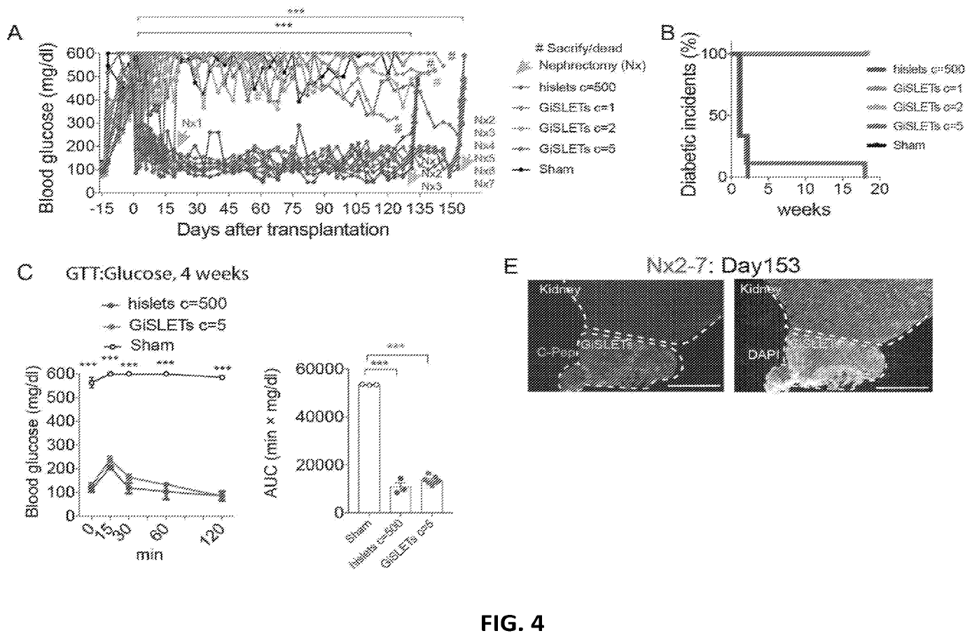

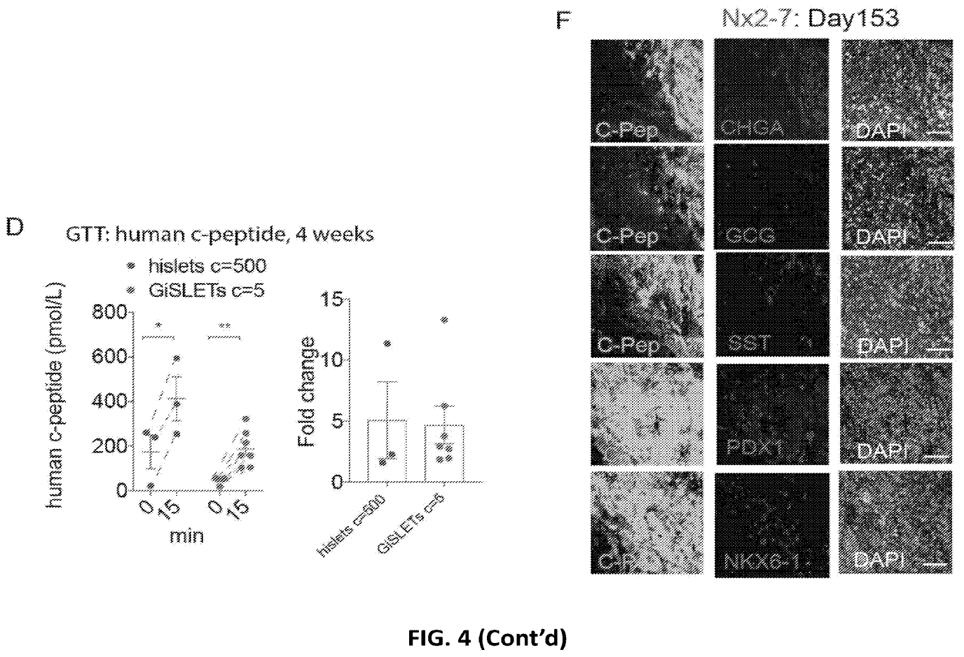

We next performed differentiation of GB-hPSCs into endoderm, mesoderm, and ectoderm lineages (FIG. 4E). Undifferentiated GB-hPSCs exhibited expression of the pluripotent stem cell marker NANOG while the endoderm marker SOX17, mesoderm marker MSX1, and ectoderm marker SOX1 were induced in individual differentiated GB-hPSCs (FIG. 6F). Endoderm marker GATA4, mesoderm marker Brachyury, and ectoderm marker OCT2 were also found in the nucleus of individual differentiated GB-hPSCs (FIG. 6G). The results revealed that GB-hPSCs possess the potential to differentiate into three germ layers.

Next, we performed step-by-step small chemical-induced islet differentiation of GB-hPSCs, by applying our developed protocol of hPSC-derived human islet-like organoids (HILOs) differentiation for mimicking human islets organogenesis (FIG. 1C). To investigate the effect of GB in human islet organogenesis, we performed bulk RNA-sequencing on day 0, 13, 22, and 30 of HILOs or GiSLETs along with hislets. Pearson's correlation across the samples showed the correlation score in the transcriptome of both HILOs and GiSLETs to be increased to that of hislets during differentiation (FIG. 1D). In addition, Pearson's correlation analysis revealed advanced differentiation of GiSLETs on day 13 (pancreatic progenitor stage), 22 (immature islets stage), and 30 (advanced maturation stage) than that of HILOs (FIG. 1D).

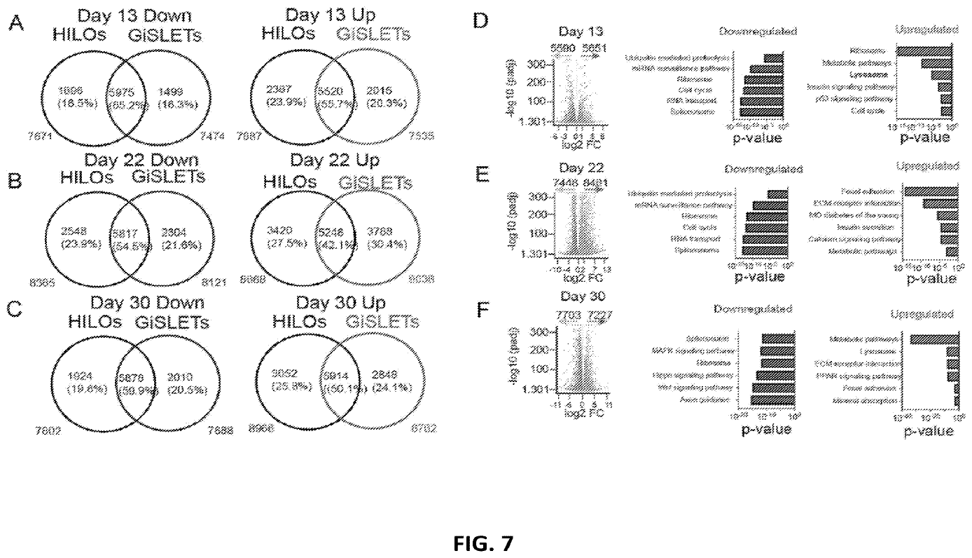

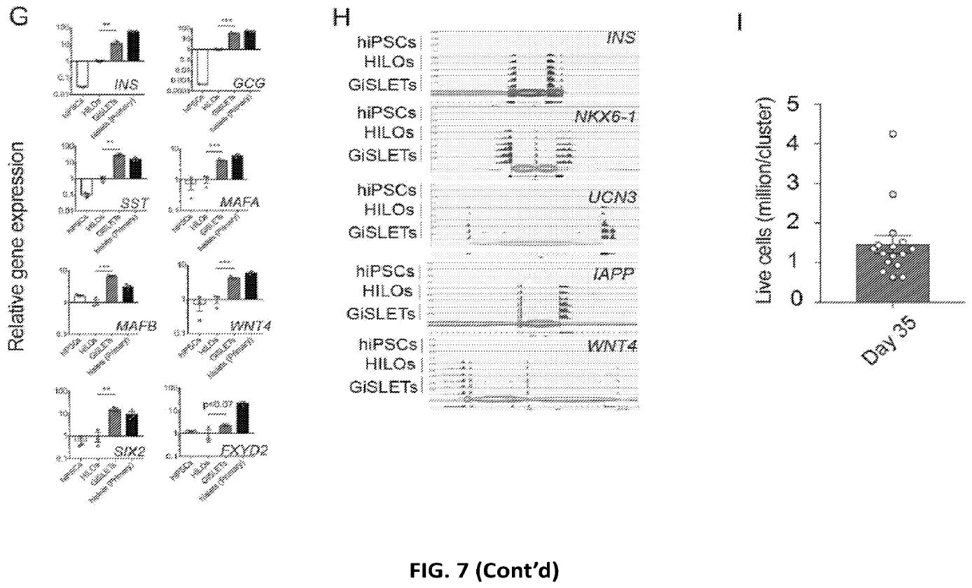

We found that 7671 and 7474 genes on day 13, 8365 and 8121 genes on day 22, and 7802 and 7888 genes on day 30 were downregulated in HILOs and GiSLETs, respectively, compared to those in hPSCs (FIG. 7A-C). Of those, 65.2% of genes on day 13, 54.5% of genes on day 22, and 59.9% of genes on day 30 were commonly downregulated. We found that 7887 and 7535 genes on day 13, 8668 and 9036 genes on day 22 and 8966 and 8762 genes on day 30 were upregulated in HILOs and GiSLETs than in hPSCs (FIG. 7A-C). Of those, 55.7% of genes at day 13, 42.1% of genes at day 22 and 50.1% of genes at day 30 was commonly upregulated (FIG. 7A-C). Next, we analyzed the subset of genes that was differentially expressed in the GiSLETs and HILOs on the same differentiated days. We found that 5651 and 5590 genes on day 13, 8461 and 7448 genes on day 22, and 7227 and 7703 genes on day 30 were upregulated and downregulated, respectively in GiSLETs than in HILOs (FIG. 7D-F). Gene Ontology (GO) analyses revealed that cell cycle-related genes as well as endocrine progenitor marker genes, such as NGN3 were transiently upregulated on day 13 of GiSLETs than of HILOs (FIG. 1E-F, FIG. 7D). Furthermore, upregulation of metabolic pathway was observed in day 22 and day 30 of GISLETs (FIG. 1F). Notably, the key β cell lineage-specific genes, such as INS, NKX6-1, UCN3, IAPP, and WNT4 were more enriched in GiSLETs than in HILOs on day 30 of differentiation (FIG. 1F, FIG. 7G, H).

Consistent with the transcriptome analyses, GiSLETs derived from both hiPSCs and hESCs sustained the larger volume of mass better than hislets or HILOs with insulin expression visualized by human insulin promoter-driven GFP (FIG. 1G). Dithizone staining of GiSLETs revealed that most of the interior of the insulin producing cells were stained intense red, suggesting that the zinc-enriched insulin producing cells survived (FIG. 1G). We found that GB-hPSCs can differentiate to giant (2,000 μm-10,000 μm in diameter) islet organoids (GiSLETs) with approximately 1.35×10⁶ surviving cells/cluster (FIG. 1G, H and FIG. 7I). These results demonstrate that GiSLETs have more enhanced islet differentiation and maturation than HILOs.

Physiological Functional Insulin Secretion from GiSLETs

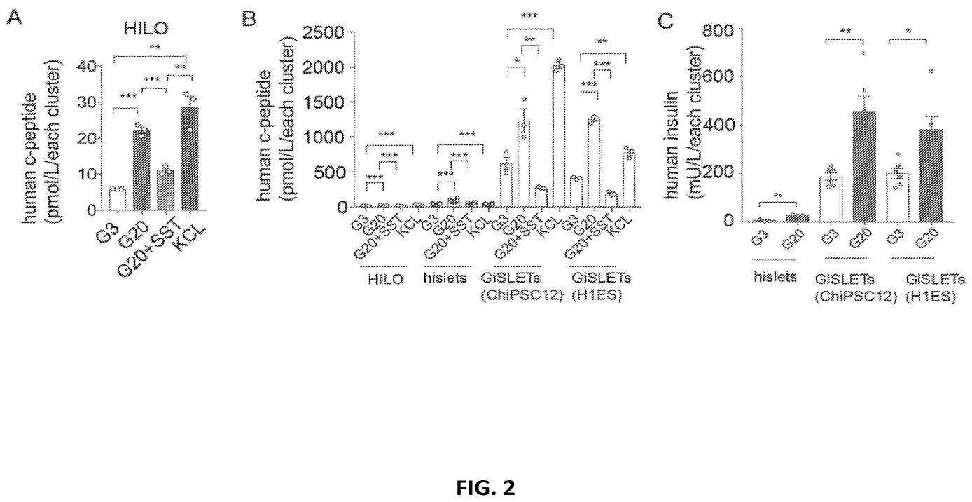

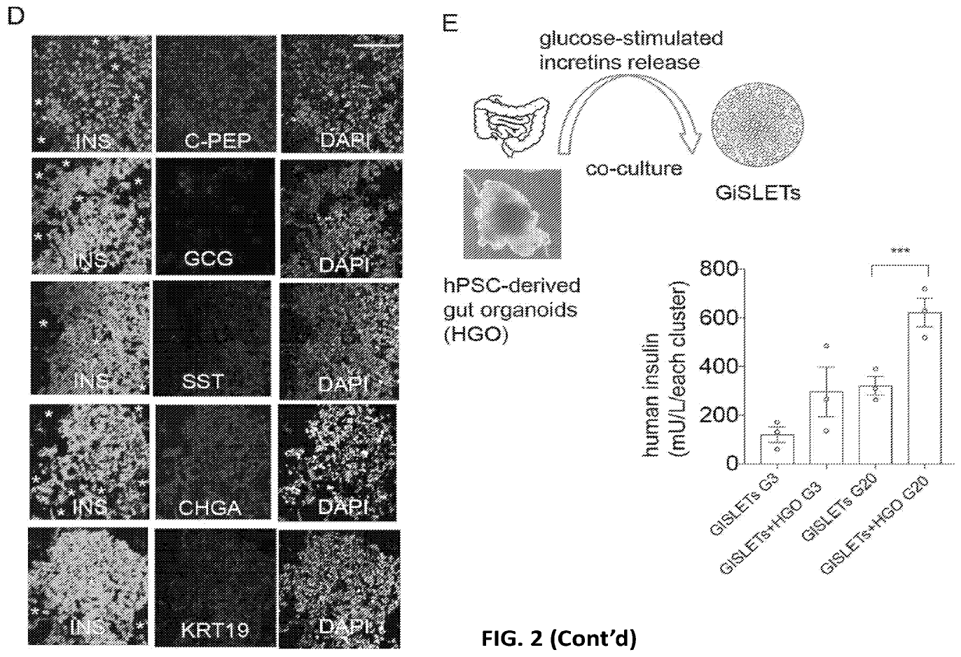

Since the volume of GiSLET is approximately ~100 times (10 times length×10 times width) larger than that of hislets or HILOs, we assumed that each GiSLET cluster can produce larger amount of insulin than each hislets or HILO. To verify the idea, we compared the insulin secretion function of GiSLETs, HILOs and hislets by measuring secreted human c-peptide, a surrogated marker for insulin, and secreted mature insulin. Both HILOs and GiSLETs derived from human induced pluripotent stem cells (hiPSCs) or human embryonic stem cells (hESCs) as well as hislets were found to exhibit glucose-stimulated insulin secretion (GSIS) function and KCl-induced insulin secretion in vitro (FIG. 2A-C). In addition, HILOs and GiSLETs showed functional somatostatin-suppressed insulin secretion (SSIS), which is a counter-regulatory paracrine pathway from δ cells to functional β cells (FIG. 2B). Notably, the total amount of c-peptide/insulin secretion from each GiSLET was approximately ~100 times more than that of each hislet or HILO (FIG. 2B, C).

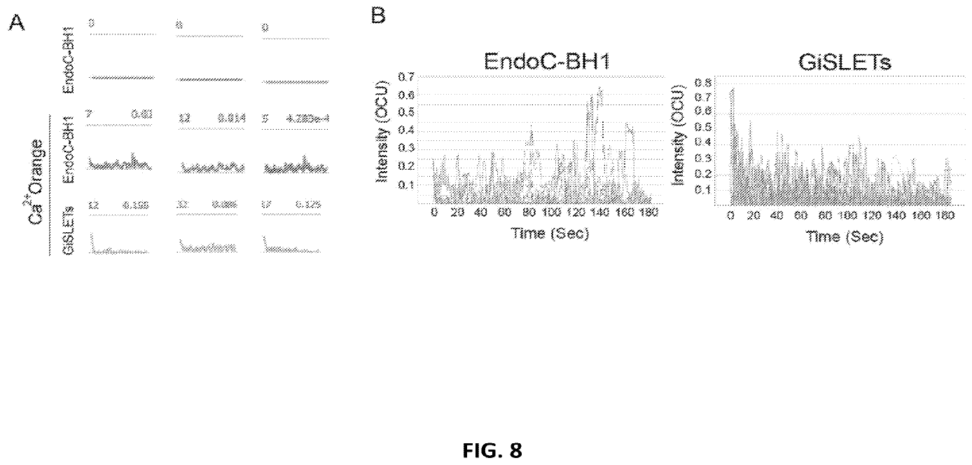

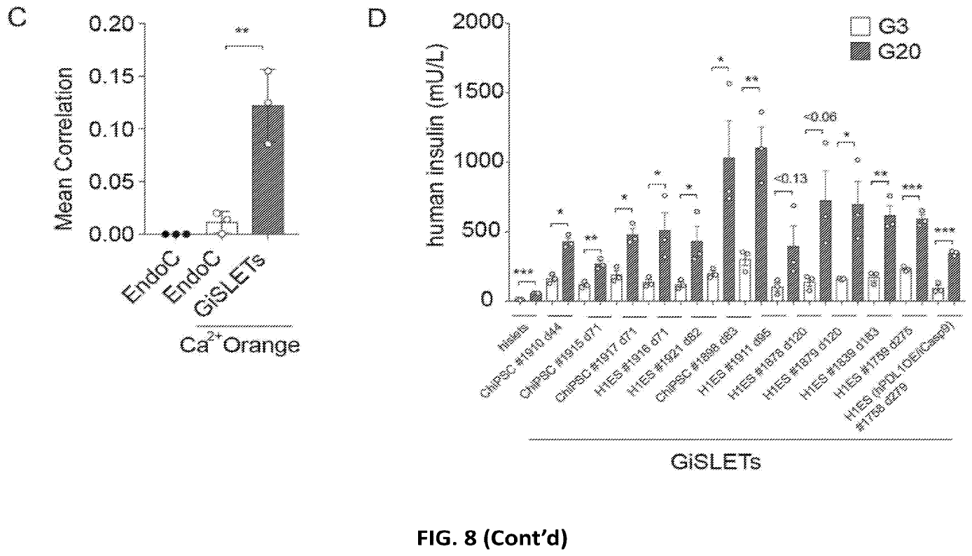

GiSLETs and EndoC-BH1 cells (a known mono-layer human β cell line, were loaded with calcium orange to detect high glucose-induced intracellular Ca²⁺ influx and oscillation. Continuous time-lapse image-based analysis by Incucyte SX5 captured the changes in intracellular Ca²⁺ flux under high glucose exposure in both GiSLETs and EndoC-BH1 cells (FIG. 8A). Intriguingly, Ca²⁺ loaded cells in GiSLETs showed higher synchronization of the oscillation of Ca²⁺ than EndoC-BH1 cells (FIG. 8B, C). The results show that encapsulated β-like cells in GiSLETs can possess in vivo-like functionality. Immunohistochemistry (IHC) study revealed the expression of key islet proteins, such as GCG (α cell), SST (δ cell), KRT19 (duct cell) expression as well as the broader expression of INS (β cell) and CHGA (endocrine) (FIG. 2D). We observed small empty spaces in GiSLETs, which might provide the gel-niche to enhance the circulation of nutrients and oxygen (FIG. 2D).

A whole-body metabolism is regulated by the cooperative physiology of multi-organ interaction. We demonstrated that GiSLETs can be co-cultured with hPSC-derived gut organoids (HGO), which secrete incretins (GLP-1, GIP, etc.) that are known enhancers of endogenous functional β cell survival and insulin secretion function. The co-culture of GiSLETs and HGO enabled the recapturing of organ-organ communication between gut and islets, characterized by enhanced GSIS function (FIG. 2E). We further investigated whether GISLETs could sustain the functionality over longer time periods than typical hPSC-derived islets. We found that GiSLETs generated from ChiPSC12, H1ESC, and genetically modified HIESC, which has incorporated genes for immune evasion (PD-L1) and induced apoptosis system (iCasp9), survived and preserved GSIS function up to 279 days (FIG. 8D). The results revealed that GiSLET technology has potential for mass production of functional insulin producing cells.

### Spatio-Temporal Gene Expression of GISLETs

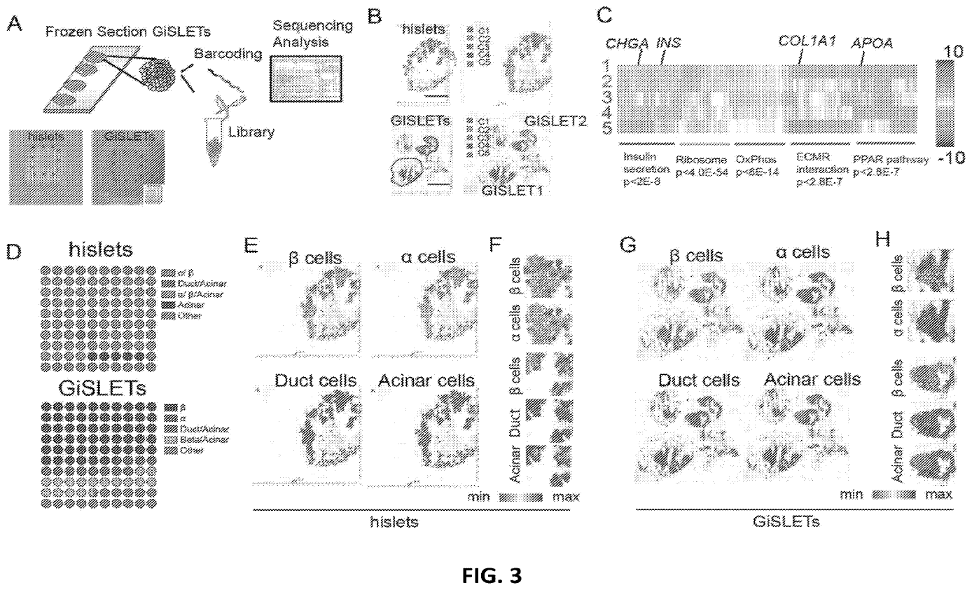

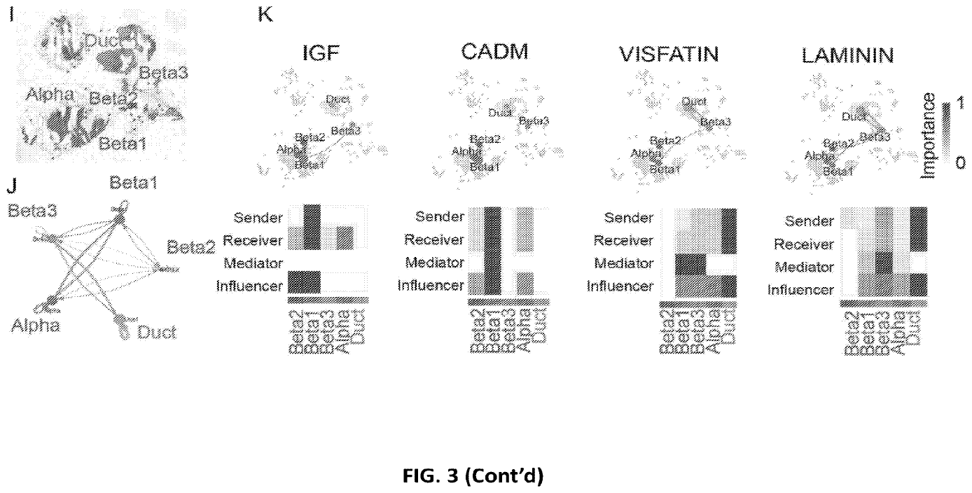

Given the larger physical size of GiSLETs compared to that of traditional stem cell-derived islets or hislets, we hypothesized that the GiSLETs technology may offer better spatial wide information for human pancreas organogenesis. To test this idea, we performed spatial transcriptome (ST) by using Visium spatial gene expression analysis (10× genomics) in GiSLETs and hislets (FIG. 3A). Larger cell cluster cross sections were obtained in GiSLETs than in hislets (FIG. 3B). Principal component analyses (PCA) and uniform manifold approximation and projection (UMAP) for dimension reduction enabled us to identify 5 clusters featuring gene expression in hislets or GiSLETs (GISLET1 & GISLET2) (FIG. 3B, C). All 5 clusters expressed INS gene (a key β cell marker), although the most enrichment of INS gene expression was observed in cluster 1 of GiSLETs (FIG. 3C).

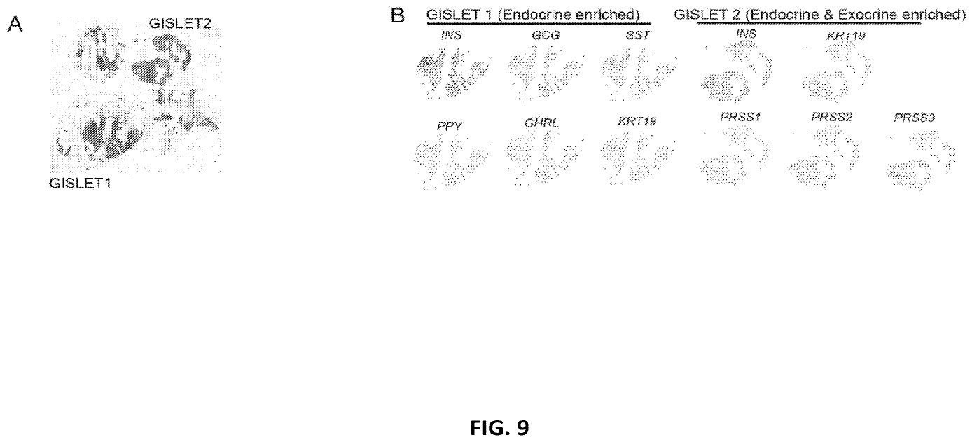

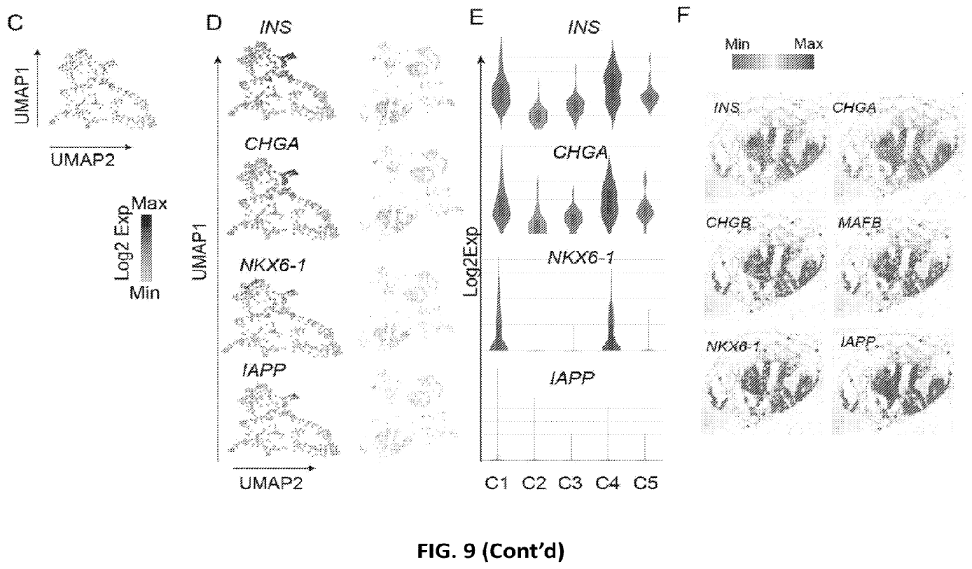

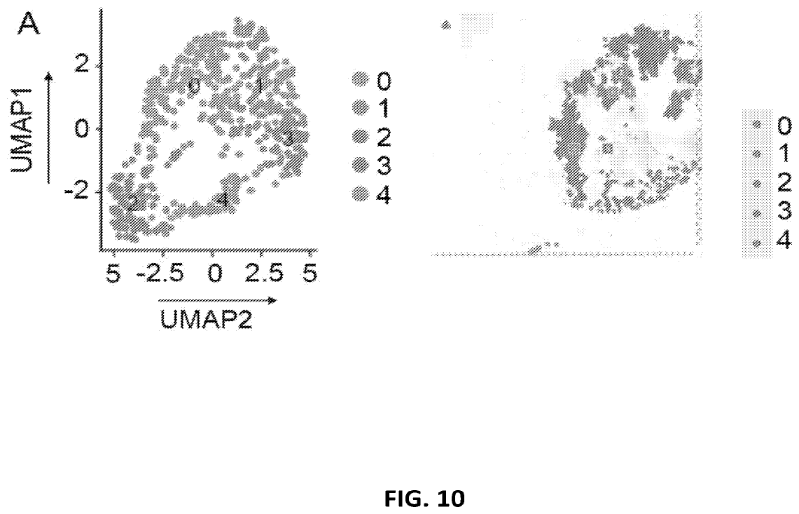

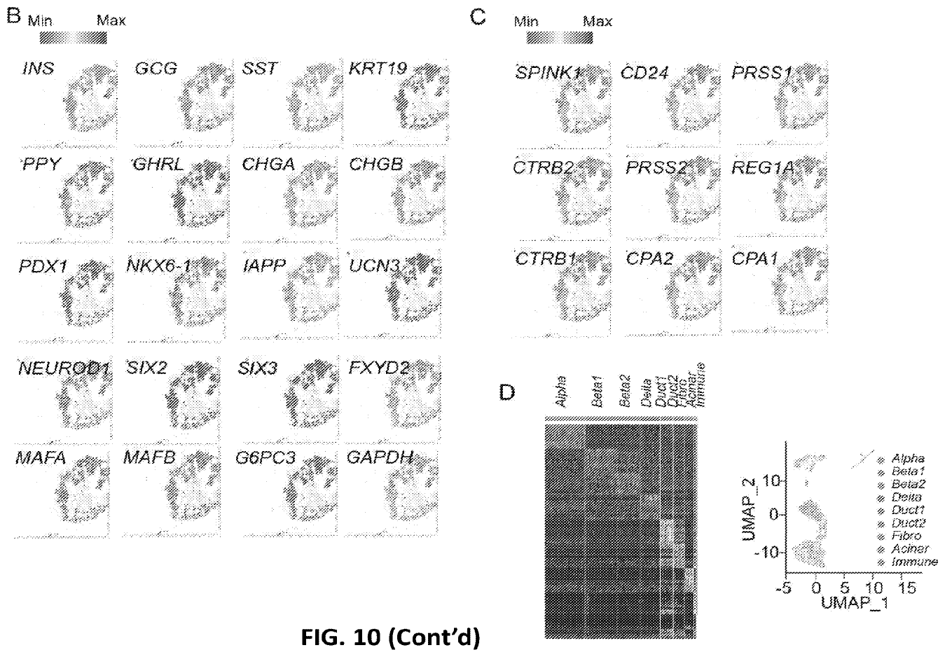

We identified the spatially restricted islet cell type-specific gene expression in GISLET1 including GCG (α cell), SST (δ cell), PPY (γ cell), GHRL (ε cell), and KRT19 (duct cell) (FIG. 9A, B). In addition, we identified the spatially restricted pancreatic exocrine-specific gene expression in GISLET2, including PRSS1, PRSS2, and PRSS3, together with INS gene expression (FIG. 9B). The cell type-specific gene expression was observed collectively in the proximal region of each cell type-specific clusters rather than as a random distribution, suggesting that during human islet development, proximal cells may influence their neighborhood cell's fate determination. In support of the notion, UMAP and Violin plot analysis revealed that key β cell/islet differentiation markers (INS, CHGA, NKX6-1, IAPP) were more enriched in cluster 1 and cluster 4 (FIG. 9C-F). In contrast, these spatially restricted gene expressions could not be clearly distinguished in hislet samples with spatially resolved transcriptional profiling at 55 μm resolution (FIG. 10A-C).

Integration of single-cell RNA-sequencing data in ST is a powerful tool to elucidate intercellular tissue dynamics. We integrated the previously generated hislet scRNA-seq data (Yoshihara et al., Nature 586, 606-611 (2020)), which contained human islet cell type transcriptome such as α cell, β cell, δ cell, duct, fibroblast, acinar, and islet resident immune cells (FIG. 10D). Based on the predicted cell type scores for each spot of spatial information, we were not able to distinguish β cell and α cell regions in primary human islets and observed the spots expressing α cell/β cell (~62%), duct/acinar cells (~2%), and α cell/β cell/acinar cell (~19%) (FIG. 3D, E, F). We found β cell (~57%) and a cell (~4%) regions to be more clearly distinguished in the cluster of GISLET1 and β cells (~57%), and ductal/acinar (~8%) regions to be more clearly distinguished in the cluster of GISLET2, thereby suggesting that dynamic spatiotemporal transcriptome can be captured in GiSLETs (FIG. 3D, G, H).

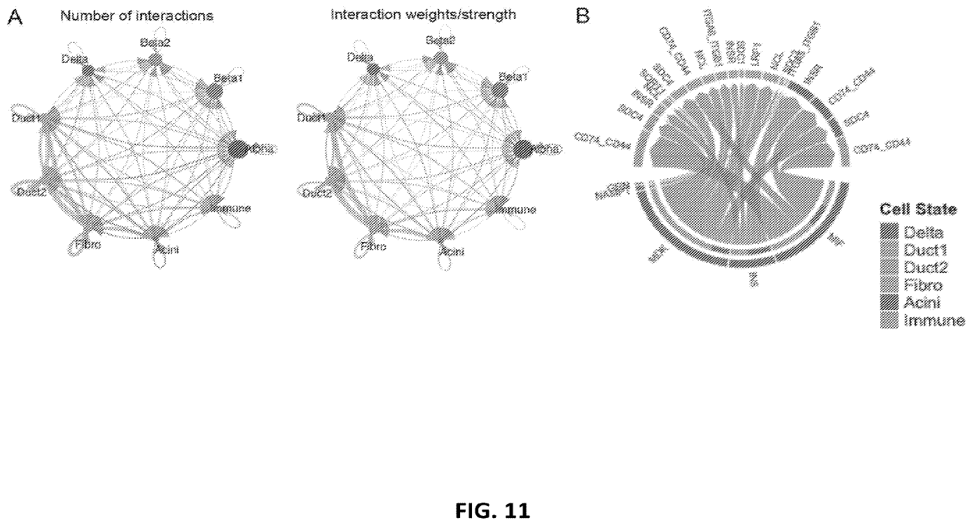

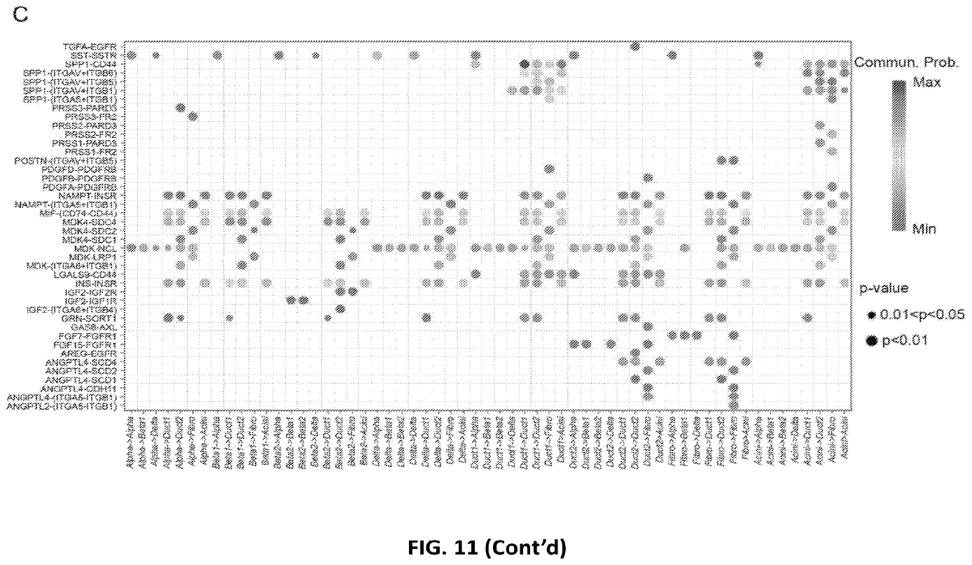

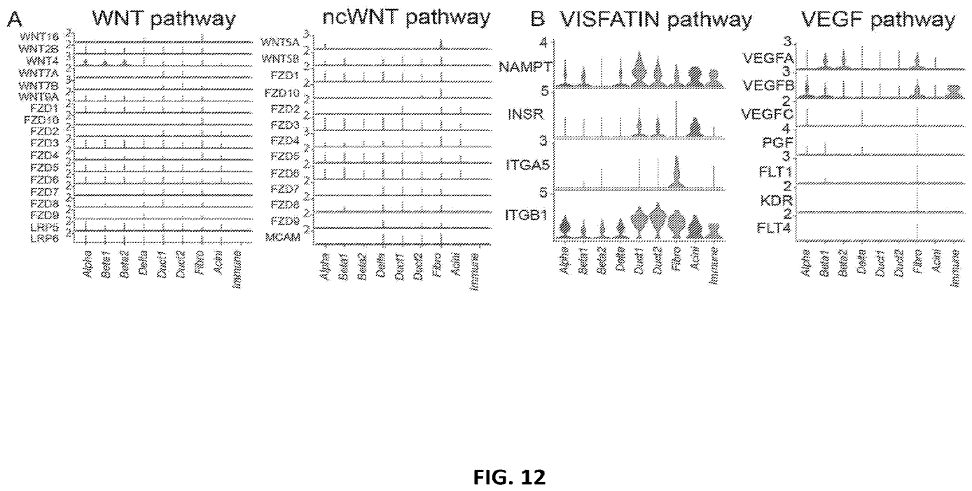

Cell-cell communication (CCC) analysis based on cross-referencing ligand-receptor interaction offers unprecedented opportunity to explore the spatiotemporal cellular organization and function. To identify the potential interactions in human islet cell types, we generated a cell-cell communication network using scRNA-seq data set of hislets along with CellChat (FIG. 11A-C). Among the cell types in hislets that we had identified (contained Alpha, Beta1, Beta2, Delta, Duct1, Duct2, Fibro, Acini, and immune), we found 42 significant ligand-receptor interaction (FIG. 11C). Given the fact that islet cells are located proximal to each other, the results indicated that CCC may significantly interfere with hislet function or development. WNT4 was enriched in β cells, and WNT5A was enriched in fibroblasts and WNT receptors Frizzled (FZDs), expressed widely in hislets, thereby suggesting that WNT and noncanonical WNT (ncWNT) pathways might act in both autocrine and paracrine manner across the hislets (FIG. 12A). Higher VISFATIN pathway enrichment was observed in Duct1, Duct2, Acini and Fibro and higher VEGF pathway enrichment was observed in Alpha, Beta1, Beta2, and Acini (FIG. 12B). Pattern recognition analysis further revealed that islet cell types jointly coordinate outgoing and incoming signals for ECM-receptor, INSULIN, IGF, FGF, and MIF pathways. We found Duct1, Duct2 and Fibro to dominantly drives signaling related to ECM-receptor (FIG. 12C-E).

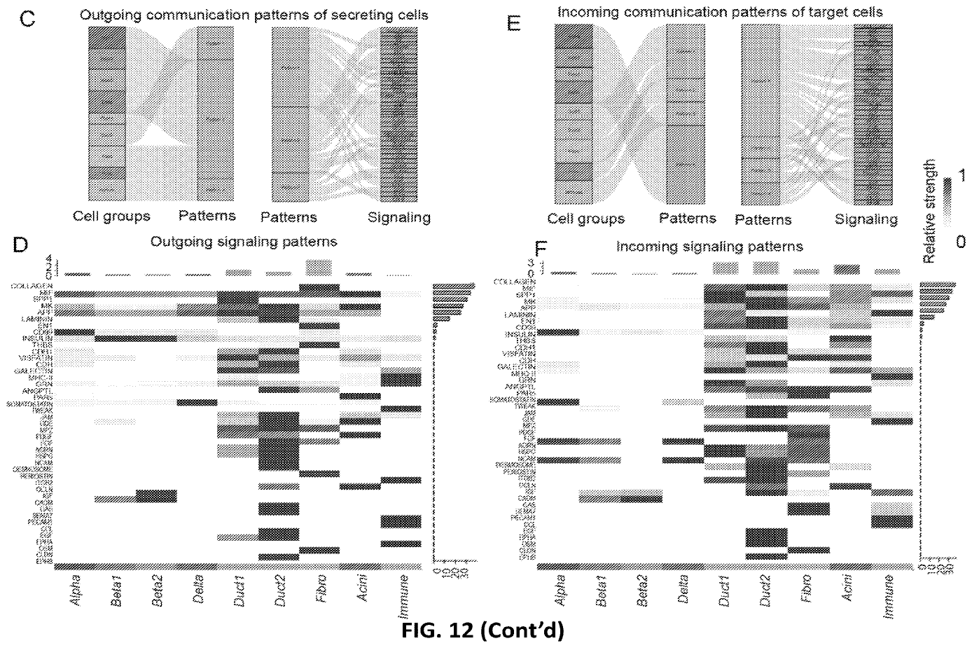

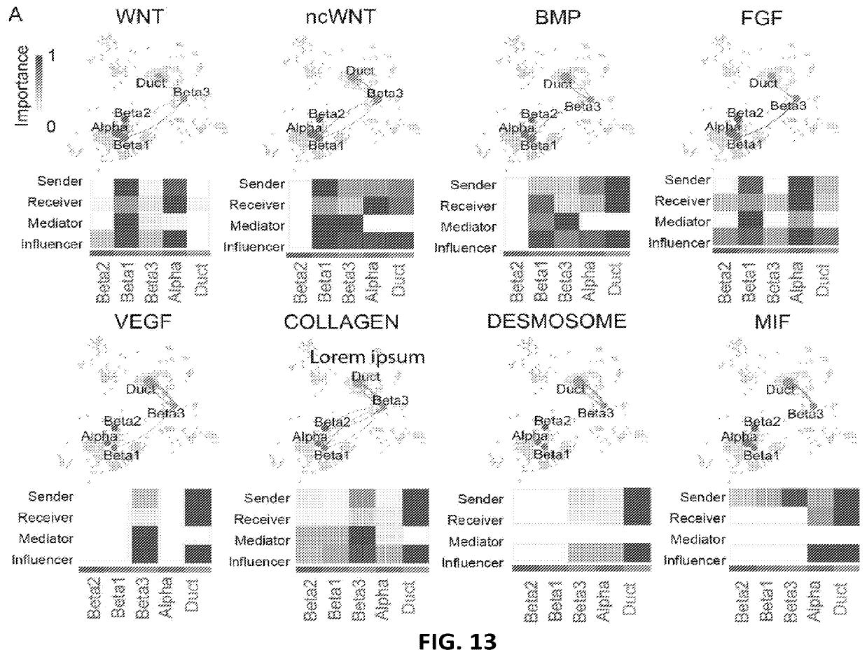

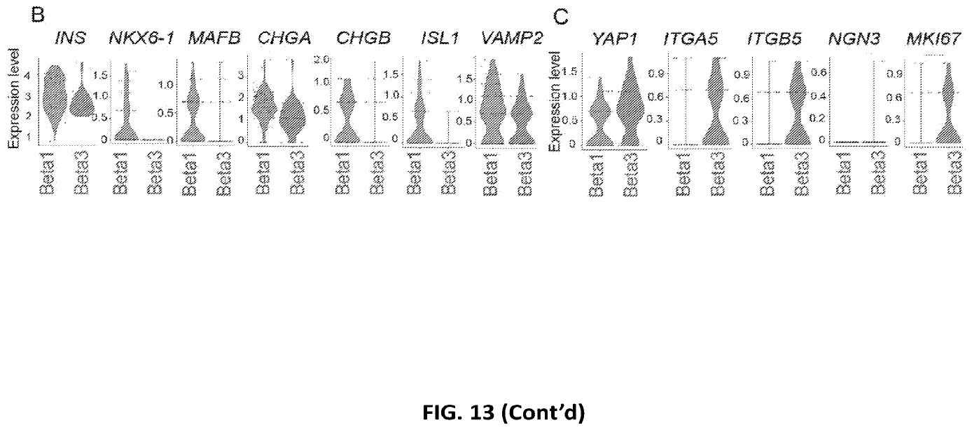

To further identify the potential spatial cell clusters communication, we applied COMMOT (COMMunication analysis by Optimal Transport) analyses in GiSLETs. We re-labeled 5 clusters that showed significantly different features of transcriptome as Beta1, Beta2, Beta3, Alpha and Duct, following their transcriptional features of pancreatic β cells, a cells, and duct cells (FIG. 3B, I). We found significant CCC across Beta1, Beta2, Beta3, Alpha and Duct of GiSLETs (FIG. 3J). Interestingly, greater weight of interaction was observed between Beta1 and Alpha or Beta3 and Duct, since those are proximally located (FIG. 3J). The inferred ligand-receptor communication network from signaling pathway analyses revealed that IGF and CADM signal pathways are enriched for CCC between Beta1 and Alpha, whereas WNT, ncWNT, BMP, and FGF signal pathways are enriched for CCC between Beta1 and Beta3 (FIG. 3K, FIG. 13A). In contrast, VISFATIN, LAMININ, VEGF, COLLAGEN, DESMOSOM, and MIF pathways, which are known extracellular matrix (ECM) modulators, were enriched for CCC between Beta3 and Duct (FIG. 3K, FIG. 13A).

We contemplated that the differential CCC in Beta1 and Beta3 may influence cellular function of β cells. YAP signaling, which is activated by ECM stiffening, was shown to inhibit pancreatic differentiation. We found YAP signal-related genes to be upregulated in Beta3 while key β cell-specific genes were downregulated in Beta3 compared to that in Beta1 (FIG. 13B, C). The results demonstrated that CCC interferes more strongly in proximally located cell clusters than distally located clusters during human islet development, and that GISLETs technology can provide fine-tuning of spatially restricted transcriptome in human islet organogenesis. It was also highlighted that GB-hiPSC-derived GiSLETs are not composed of a numbers of small islet clusters, rather of a larger mass of human islet cluster.

### GISLETs Ameliorate Preexisting Diabetes in NOD-SCID Mice

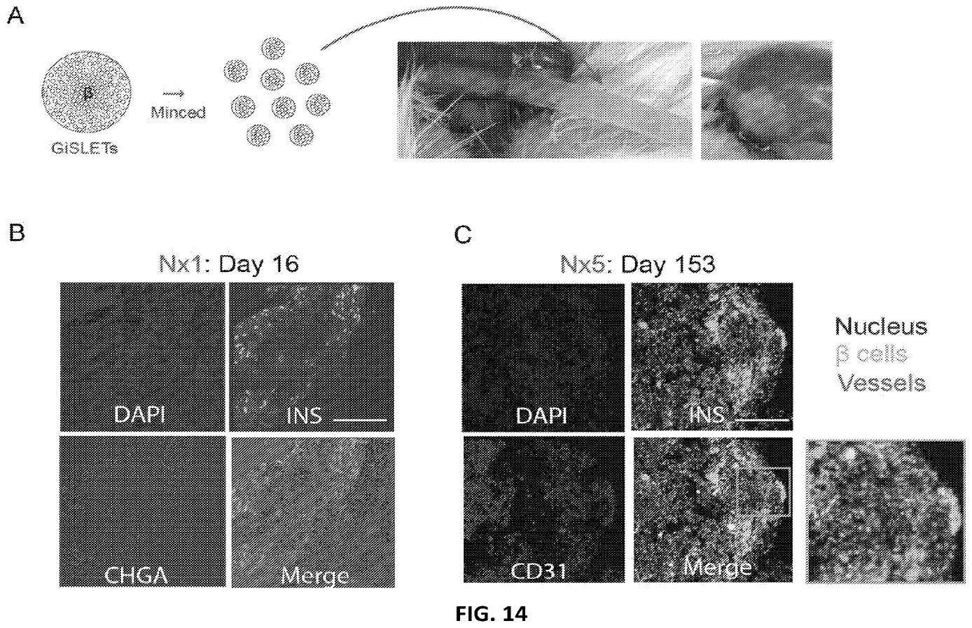

To investigate whether GiSLETs have the functional efficacy to cure diabetes in vivo, we transplanted GiSLETs in streptozotocin (STZ)-induced diabetic mice under immune-deficient condition (NOD-SCID mice). Since GiSLETs are supported by GB, they are easily morphologically transformed to fit the highly vascularized but narrow transplantation sites. To enhance the potential vasculature innervation, we minced GiSLETs into a smaller size for a transplantation study (FIG. 14A). We found that, not 1 or 2 but 5 clusters of GiSLETs, transplanted through kidney capsule reduced blood glucose to the normal range (80-250 mg/dl) within a few weeks after transplantation demonstrated successful early graft survival (FIG. 4A, FIG. 14B). Notably, 5 clusters of GiSLETs showed more than 150 days of blood glucose control, an efficacy similar to that of 500 clusters of hislets (FIG. 4A). We observed that nephrectomy (Nx; graft removal) performed on day 153 post transplantation, to remove the transplanted grafts, resulted in rapid loss of glycemic control and reestablishment of hyperglycemia, which confirmed the transplanted GiSLETs sustained normal glycemia in vivo (FIG. 4A). We observed approximately 90% of mice to have normalized glucose within a few weeks of post transplantation (FIG. 4B). IHC analyses revealed INS/CHGA-positive early graft survival on day 16 after the transplantation. We performed intraperitoneal glucose tolerance (i.p. GTT) test to assess the glucose disposal kinetics and GSIS in vivo in the mice receiving transplanted human islets or GiSLETs at 4 weeks after transplantation. The i.p. GTT test showed improved glucose clearance by both hislets and GISLETs transplantation (FIG. 4C). In vivo, glucose-stimulated insulin/c-peptide secretion was observed in the mice receiving hislets or GiSLETs (FIG. 4D). IHC analysis of GiSLETs graft on day 153 after transplantation revealed that c-peptide-positive β-like cells co-expressed critical β cells markers, such as CHGA, PDX1, and NKX6-1 while a few monohormonal GCG- or SST-positive cells were also observed (FIG. 4E, 4F). CD31-positive vascular cells were well innervated in the area of GiSLET graft on day 153 after the transplantation (FIG. 14C). Furthermore, there was no evidence of GG remaining in the transplantation sites, suggesting the biodegradation of the gels with no significant adverse physiological impact in the recipient mice (FIG. 14C). No teratoma formation was observed within 153 days of after the transplantation.

Taken together, our results demonstrate that the GiSLET technology could provide a novel alternative method for mass production of functional β cells for long-term effective therapy for diabetes.

### Giant Organoids System Recaptures Intestine-Islet Communications In Vitro

A whole-body metabolism is regulated by the corporative physiology of multi-organ interaction. We have demonstrated that GiSLETs can be co-cultured with hPSC-derived gut organoids (HGO), which secretes incretins (GLP-1, GIP etc) as known enhancer of endogenous functional β cell survival and insulin secretion function (FIG. 2E). The co-culturing of GiSLETs and HGO enabled recapturing organ-organ communication between gut and human islets, characterized enhanced GSIS function (FIG. 2E). We have also demonstrated that gellan-gum encapsulation is applicable for other type of cells such as hPSC-derived cardiomyocyte to generate larger size of organoids. These result highlight the potential of GiSLETs technology for mass production of cells, fine-tuning spatial transcriptome analyses to dissect the mechanism of human development and organ-organ communication.

### Material and Methods

### Cell Culture

### Preparation of GB-Stem Cells

### Common Reagents:

1. 1% (w/v) and 0.3% (w/v) Gellangum (low acyl GG GG-LA: Modernist pantry). Dissolve Kelcogel (KG) F in H₂O by stirring at 90 °C (i.e. 1 g/100 mL) or autoclave at 121 °C for 20 min Store Kelcogel F at 4 °C.
2. 3% Methylcellulose (MC, R&D systems)
3. Y27632 (ROCK inhibitor, STEM core; Stock: 10 mM)
4. 3D mTeSR Plus Media: 0.015% Kelcogel F+0.009% MC stock solution+10 μM ROCK inhibitor e.g. 500 mL mTeSR Plus+25 mL 0.3% Kelcogel+1.5 mL MC stock solution (+1:1000 dilution of ROCK inhibitor, fresh, after each refeeding)+5 mL Penicillin/Streptomycin (P/S) (GIBCO, 15-140-122)
Differentiation Media for stages S1-S5: see Appendix 1
e.g. 500 mL MCDB131 (S1-S5)+25 mL 0.3% Kelcogel+1.5 mL MC stock solution
5. Matrigel: Cultrex® Reduced Growth Factor Basement Membrane Matrix (Travigen, 3433-010-01)
6. DPBS (Ca²⁺): GIBCO 14040-133
   Day Before Culturing hPSCs:
1. Prepare feeder-free Matrigel coated 6-well plates:
   a. Collect 1 mg of aliquoted Matrigel from −80 °C and place on ice.
   b. Aliquot 24 mL of cold DMEM-F12 into a 50 mL tube.
   c. Collect 1 mL of DMEM-F12 from the 50 mL tube and resuspend the Matrigel aliquot. Transfer the resuspended Matrigel solution to the 50 mL tube, and mix.
   d. Seed 2 mL into each well in a 6-well plate (1 mg Matrigel makes 2× 6-well plates).
   e. Store Matrigel 6-well plates in 37 °C incubator.
      Proliferation of hPSC Clusters (5-7 days):
1. Culture hiPSCs (or hPSCs or hESCs) on Matrigel coated 6-well plates with mTeSR Plus (StemCell Technologies, 100-0276). Check the cells daily for spontaneous differentiation and feed cells. Passage when ~70% confluent.
   1. Passage using ReLeSR method:
      a. Mark areas of differentiation using the objective marker in the microscope.
      b. Aspirate media and aspirate the marked areas of differentiation in the well (3-4 taps/differentiated area).
      c. Add 0.5 mL of ReLeSR (STEMCell Technologies, 05872) to each well and incubate at R.T. for 5 mins (longer incubation based on cell density).
      d. During the incubation, aspirate the DMEM-F12 from the previously coated 6-well matrigel plate and add 2 mL of mTeSR Plus media per well.
      e. After incubation, check the cells under a microscope—cells should appear shiny with slightly curled edges around the colonies, but still attached to the well.
      f. Aspirate ReLeSR from the well without disturbing the attached cell layer.
      g. Using a 10 mL glass pipette, gently pipette/scratch the media with cells in a back and forth motion 3-4 times or more until you collect all the cells.
      h. Split cells between 1:6~12.
      i. Place the cells in the incubator and gently move the plate back and forth and side by side in a plus motion.
      j. Cells generally need to be passaged 4-6 days. Change media every other day. (e.g. Mon 2 ml/well, Wed 2 ml/well, Fri 4 ml/well)

### GB-Stem Cell Clustering-Differentiation

1. Aspirate culture medium from cultures
2. Add 0.5 mL accutase to each well; wait 5 minutes
3. Wash with 0.5 mL PBS in each well. Make sure to wash off all the cells. Collect in a 50 mL culture tube.
4. Centrifuge the 50 mL culture tube at 1300 rpm for 5 minutes.
5. Aspirate the PBS and accutase, leaving only the cell pellet. Cell count.
6. Dilute the cell pellet to 1~10 million cells per 10~25 μL with 1% KG and mix gently.
7. Use a pipette to slowly drop in 10~25 μL GB-Stem into Ca²⁺ containing PBS (GIBCO, 14040-133) with 0.1% KG. Very slowly eject the mixture. 37 d ~5 min incubation.
▴Note: Both 1% KG and Ca²⁺ containing PBS need to be pre-warmed to 37 °C in water bath.
8. Add 3 mL mTeSR Plus Basal Medium (Component #100-0274 and 10 μM final concentration of Rocki (3 μL in this case) with or without recombinant FGF2 (10 ng/ml). Culturing 0~7 days (Media change every other day) prior differentiation.
▴Note: IF GB-Stem survives, the color of culture media changes to more orange to yellow next day.
9. Differentiate to GISLETs by following Table 1 chemical combination with 70 rpm orbital shaking at 37 °C, 5% CO₂ condition.

### Mice Diabetes Model

Hyperglycemia (>400 mg/dl blood glucose) were induced by injecting 1 dose of 180 mg/kg STZ in all the mice prior of GISLETs transplantation.

TABLE 1
GISLETs differentiation: Timing of Differentiation Media
Changes Basic culture condition 3 ml of differentiation
media/6well with 60-70 rpm orbital shaking

| | 2D or | |
|---|---|---|
| | 3D | |
| Day | Media | Supplements |
| 0* | S1 | rhActivin A (100 ng/ml), CHIR99021 (3 μM) |
| 1 | | rhActivin A (100 ng/ml) |
| 3* | S2 | rhKGF (50 ng/ml) |
| 5* | S3 | rhKGF (50 ng/ml,) SANT-1 (0.25 μM), RA (1 μM), |
| | | LDN193189 (100 nM), Alk5iII (10 μM), |
| | | TPB (200 nM) |

Note: Monitoring glucose concentration carefully after day 5. The glucose
concentation changes withon 24 hours since the sphere use glucose as
energy for progenitor expansion. 8 mM glucose concentration can be
dropped down <5 mM. If the glucose lowered <5 mM add the additional
glucose at day 6, day 8, day 9, day 11 to adjust glucose concentration to
8 mM without changing whole media. Lactate production from the
spheres may oxidaze the differentiation media. If the Media color
changed to bright yellow, additional media change may be required to
remove oxidized media.

| | | |
|---|---|---|
| 7* | | rhKGF (50 ng/ml,) SANT-1 (0.25 μM), RA (100 |
| | | nM), LDN193189 (100 nM), Alk5iII (10 μM), |
| | | TPB (100 nM) |
| 9 | | (same from Day 5) If the cell loss was observed, |
| | | skip day 9 (No media change) |
| 10* | S4 | SANT-1 (0.25 μM), RA (50 nM), T3 (1 μM), |
| | | Alk5iII (10 μM),, LDN193189 (100 nM) |
| 12 | | (same from Day 10) If the cell loss was observed, |
| | | skip day 9 (No media change) |
| 13* | S5 | T3 (1 μM), Alk5iII (10 μM), GsiXX (100 nM), |
| | | LDN193189 (100 nM) |

Note: Day 13-19 Media as well as Day 20 Media can be pre-made and
Keep it in 4 d for a several weeks.

| | | |
|---|---|---|
| 15 | | (same from Day 13) |
| 17 | | (same from Day 13) |
| 19 | | (same from Day 13) If the cell loss was observed, |
| | | skip day 19 (No media change) |
| 20* | S5: | T3 (1 μM), Alk5iII (10 μM), Trolox (10 μM), R428 |
| | Immature | (2 μM), N-Cys (1 mM) (7-20 days - check |
| | β-cells | differentiation) |

Note: After D 20, qPCR analysis for hINS, hUCN3, hMAFA, hMAFB,
hNKX6-1 should be used to check the terminal differentiation.

| | | |
|---|---|---|
| 22 | | (same from Day 20) |
| 24 | | (same from Day 20) If the cell loss was observed, |
| | | skip day 24 (No media change) |
| 25* | S6: | T3 (1 μM), Alk5iII (10 μM), WNT4-Sup (50~200 |
| | Mature | ng/ml of rhWNT4 containing)(Alternatively rhWnt4 |
| | β-cells | (100 ng/ml) for 5-10 days. |
| | (mito. | |
| | mature) | |
| 27 | | (same from Day 25) |
| 29 | | (same from Day 25) |

Note: Timing of rhWNT4 maturation step flexible after D 25
differentiation. GISLETs can be cultured <100 days in final
differentiation media.

| | | |
|---|---|---|
| 31 | S7: | T3 (1 μM) |
| 33 | Maintain | (same from Day 31) |
| 35* | functional | (same from Day 31) |
| | β-cells | |

Note: The matured cells can be used for in vitro GSIS
assay and transplantation
*Check differentiation by qPCR (Appendix 3) - collect ~1 mL of suspended spheroid culture before replacing the media

Base Differentiation Medias (with 25 mL 0.3% Kelcogel+1.5 mL MC Stock Solution [in 500 mL S1-S5 Media])

Prepared media are initially stored overnight at 4 °C to confirm complete solubilization prior to sterile filtering (0.22 μm filter). Media preparations are stable for 2-4 weeks at 4 °C and 8-12 months at −20 °C.

For the addition of Gellangum, both media and Gellangum must be prewarmed at 37 °C before mixing (mix via inverting bottle). Also, media (containing Gellungum and MC) must be prewarmed before the addition of reagents/recombinants protein.
S1 Media: MCDB131 (8 mM glucose)
S2 Media: MCDB131 (8 mM glucose)
S3 Media: MCDB131 (8 mM glucose)
S4 Media: MCDB131 (8 mM glucose, Zinc sulfate, Heparin)
S5 Media: MCDB131 (20 mM glucose, Zinc sulfate, Heparin)
S6 Media: MCDB131 (20 mM glucose, Zinc sulfate, Heparin, WNT4 supernatant)

### Example 2. Identification of Functional Human Islets Derived from Stem Cells

This example identified that FXYD Domain Containing Ion Transport Regulator 2 (FXYD2) as a novel regulator of functional maturation by influencing β cell transcriptome necessary for GSIS.

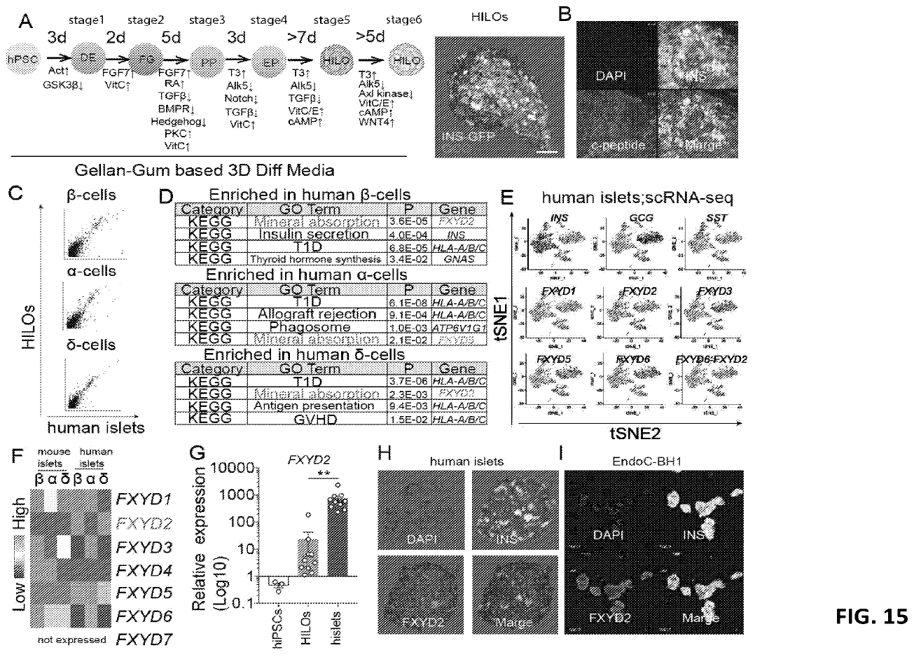

FXYD2 is restrictedly expressed in primary human β cells and δ cells, which was found as a top down-regulated gene pathway in human pluripotent stem cell (hPSC)-derived human β-like cells and δ-like cells (FIG. 15A-I). Doxycycline-induced FXYD2 expression enhances glucose-stimulated insulin secretion (GSIS) by enhancing the transcriptional pathway necessary for GSIS. This example found that FXYD2 regulates NaCl⁺ osmotic stress, which provides a novel screening method for selecting functionally mature HILOs.

Human pluripotent stem cells (hiPSCs) provide a potential alternative source of pancreatic β cells and hold the promise to cure diabetes. While current approaches generate β-like cells from hiPSCs in vitro that resemble primary β cells from cadaveric islets, these cells still lack several hallmarks of full maturity, including gene expression and mounting a proper insulin secretion response to glucose. Immature juvenile and neonate β cells undergoes postnatal functional maturation, which is the process to enhance glucose sensitivity and enhances GSIS function. The pathway for postnatal functional islet maturation, which typically takes a few years after birth in humans, is not fully understood. Therefore, identifying such a pathway is necessary to improve the immaturity of generated islets, batch-to-batch differences, and perfect control of the generated cell products.

We have identified that metabolic shifts from glycolytic toward more oxidative status enhances GSIS postnatally and can enhance the function of hiPSCs-derived β-like cells. It is important to understand the cellular diversity within the developing islet, as well as the complex cellular interactions that may influence functional differentiation of islets from hiPSCs.

Acknowledging the importance of 3-dimensional (3D) organization and cell-cell communications in organ function and the terminal differentiation of organ-specific cell types, we have adapted our technology to generate 3D-structured human pancreatic islet tissues called human islet-like organoids (HILOs) from pluripotent stem cells. Notably our HILOs contain the basic cell types necessary for proper islet function, including insulin-producing β cells, glucagon producing α cells and somatostatin producing δ cells. Although similar organoid systems have recently been established for other tissues such as intestine, kidney and brain, the matrigels typically used for organoid generation limits the scalability and economic viability. Therefore, as demonstrated in Example 1, we developed a novel organoid generation system for pancreatic islets by using a gellan-gum containing functional polymer-based 3D culture system, which is fully scalable and economical.

It has been observed that stem cell-derived islets can have lower insulin production, lower amplitude of GSIS, and slower glucose responsiveness compared to primary human islets. We identified a new functional maturation marker FXYD2, which uniquely distributed in β cells and δ cells and FXYD3/5/6 is uniquely distributed in α cells but not in those of HILOs. These findings are critical with human context since we identified FXYD3/5/6 but not FXYD2 is enriched in rodent β cells. We found that regulation of FXYD2 expression correlated to GSIS function and its links to the transcriptional alternation for human β cells led to more mature status in vitro. We found that FXYD2 expression is regulated by glucocorticoid receptor (GR) signaling, thyroid hormone receptor (TR) signaling and NaCl⁺ osmotic pressure synergistically. Further we established FXYD2-based screening of functionally mature HILOs (fHILOs).

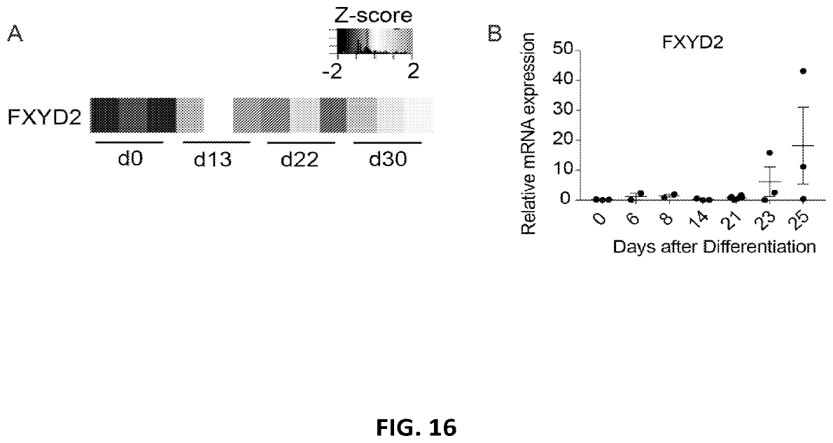

FXYD2 links mineral absorption pathway to functional maturation. We observed that the differentially expressed genes between HILOs and human islets using single cell RNA-seq (scRNA-seq) analyses. We identified FXYD2, a known sodium and potassium transporting ATPase gamma chain subunit and related mineral absorption pathway as a top down-regulated pathway in β cells and δ cells of HILOs compared to those of human primary islets (FIG. 15A-15C). FXYD genes encode a member of a family of small membrane proteins that share a 35-amino acid signature sequence domain, beginning with the sequence PFXYD and containing 7 invariant and 6 highly conserved amino acids. We found that the unique distribution of FXYDs family genes expression in human primary islets and FXYD2 expression is restricted in β cells and δ cells population in islets and no other FXYDs are expressed in human β cells (FIG. 15D, 15E). Notably we found rodent β cells predominantly express FXYD3, 5, 6 but not FXYD2, while human β cells only express FXYD2, which highlight the importance of FXYD2 function in human model rather than the rodent model (FIG. 15F). Although, we observed FXYD2 expression was increased during in vitro human islet organogenesis (FIG. 16A, 16B), consistent with the scRNA-seq results, we found that FXYD2 expression was ~100 times lower than that of human primary β cells (FIG. 15G). The majority of FXYD2 expression was observed in cellular membranes in human islet β cells and human β cell line EndoC-BH1 cells (FIG. 15H, 15I). These results suggest that FXYD2 as a novel functional β cell marker in stem cell derived β cells.

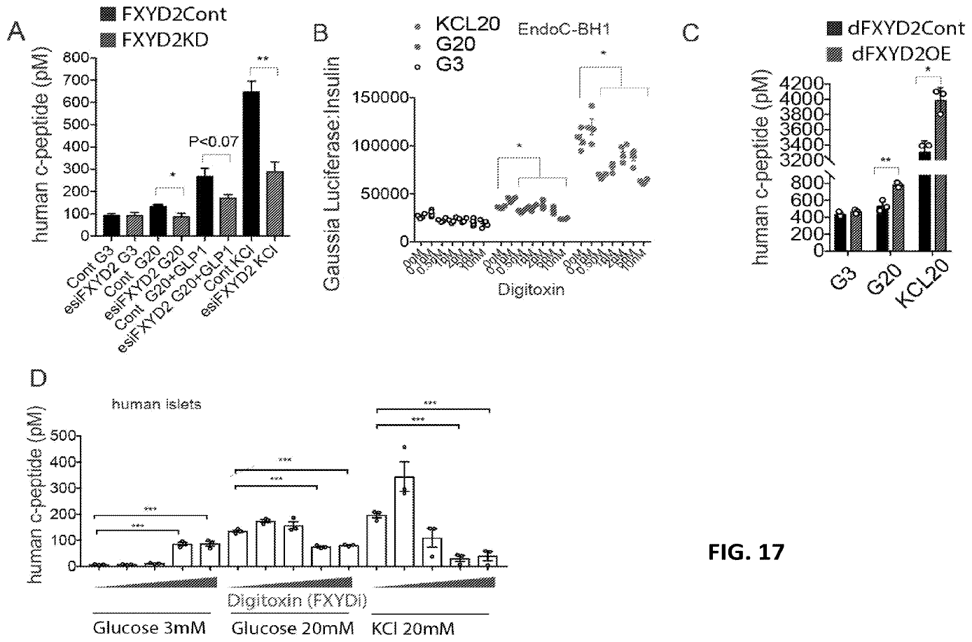

FXYD2 positively regulates insulin secretion in human β cells. We hypothesized that FXYD2 positively regulates acute GSIS function in human β cells. To identify the physiological role of FXYD2 in human β cells, we generated doxycycline (dox)-induced FXYD2 overexpression (dFXYD2OE) and knockdown (FXYD2KD) in clonal human β cell line, EndoC-BH1 cells. Human c-peptide is the stable, surrogate marker of human insulin. We found that FXYD2KD suppresses glucose, GLP-1 and KCl depolarization-stimulated c-peptide secretion in the EndoC-BH1 (FIG. 17A). We established stable EndoC-BH1 cell line with proinsulin-NanoLuc, a construct engineered to co-secrete insulin and gaussia luciferase at same molar ratio. Pharmacological FXYD2 inhibition (FXYD2i) by digitoxin suppresses GSIS and KCl-induced gaussia luciferase secretion (FIG. 17B). On the other hand, dFXYD2OE enhances glucose and KCl depolarization-stimulated c-peptide secretion in EndoC-BH1 cells (FIG. 17C). These results suggest that FXYD2 expression reciprocally regulates insulin secretion function in human β cells. Next, we wondered whether FXYD2 pathway regulate insulin secretion in multi-cellular 3D structured islets. To test this idea, we have used primary human islets. We found that pharmacological FXYD2 inhibition using digitoxin (FXYD2i) increases low glucose (3 mM) stimulated c-peptide secretion and decreases high glucose (20 mM) stimulated c-peptide secretion in primary human islets (FIG. 17D). These results suggest that FXYD2 is required for functional insulin secretion in mature human β cells.

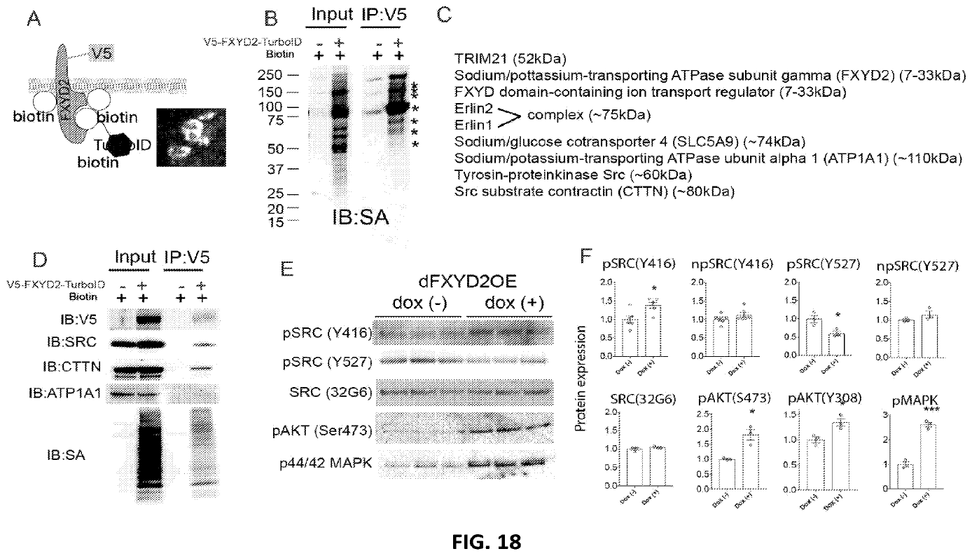

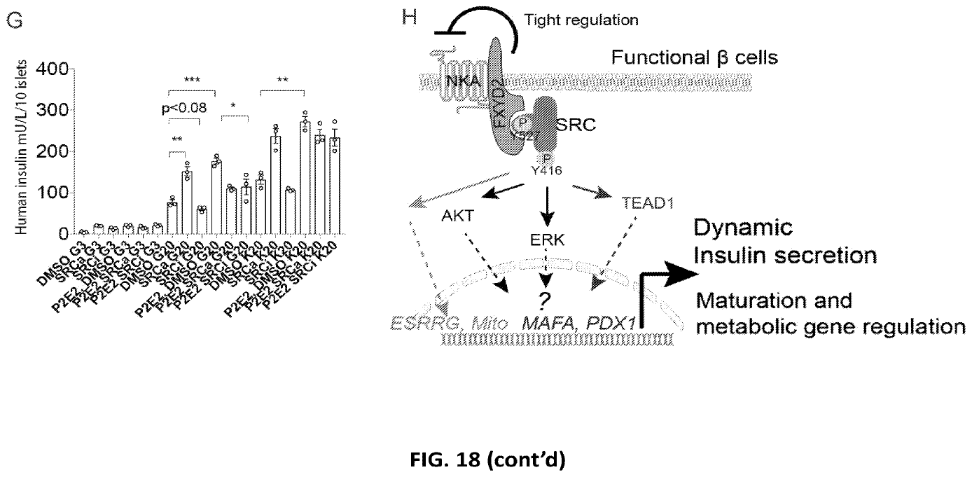

FXYD2 enhances SRC signaling dependent transcriptional pathway by physical interaction. We hypothesized that FXYD2 may regulate signal complex to regulate cellular function and remote control of gene expression. To identify the protein complex of FXYD2, we constructed the lentivirus vector containing APEX and TurboID containing FXYD2 protein proximal labeling (pLv-V5-FXYD2-APEX-EGFP and pLV-V5-FXYD2-TurboID-EGFP) (FIG. 18A). Prior of FXYD2 immunoprecipitation (IP) by V5-tag, we treated cells with 500 nM biotin for overnight to visualize FXYD2 proximal proteins. We found many biotinylated proteins, which were visualized by streptavidin antibody (SA) were in the IP samples (FIG. 18B). Next, we performed Mass-spec analyses to identify these potential FXYD2 binding proteins. Our LC-MS/MS analyses identified that FXYD2 (7-33 kDa), TRIM21 (52 kDa), Erlin1/2 (complex ~75 kDa), SLC5A9 (~74 kDa), ATP1A1 (~110 kDa), SRC (60 kDa), SRC substrate/CTTN (~80 kDa) were containing in the IP samples (FIG. 18C). We confirmed the SRC as an interacting partner of FXYD2 by IP analyses (FIG. 18D, 19A). SRC is a non-receptor cytoplasmic tyrosine kinase that becomes activated following the stimulation of plasma membrane receptors, including receptor tyrosine kinases and integrins, and is an indispensable player in multiple physiological homeostatic pathways. SRC shows reciprocal function to maintain dynamic gene expression and metabolism in normal cells and promotes tumorigenesis and metastasis in cancer cells. SRC regulates insulin secretion in β cells context dependent manner. SRC activity is regulated by tyrosine phosphorylation at two sites, but with opposing effects. While phosphorylation at Tyr416 in the activation loop of the kinase domain upregulates enzyme activity, phosphorylation at Tyr527 in the SH2 domain renders the enzyme less active. We found dox-dependent FXYD2 induction enhanced SRC phosphorylation at Tyr416 and downstream AKT pathway, while reduces SRC phosphorylation at Tyr527 (FIG. 18E, 18F). These results suggest that FXYD2 regulates the SRC signaling pathway by direct protein-protein interaction.

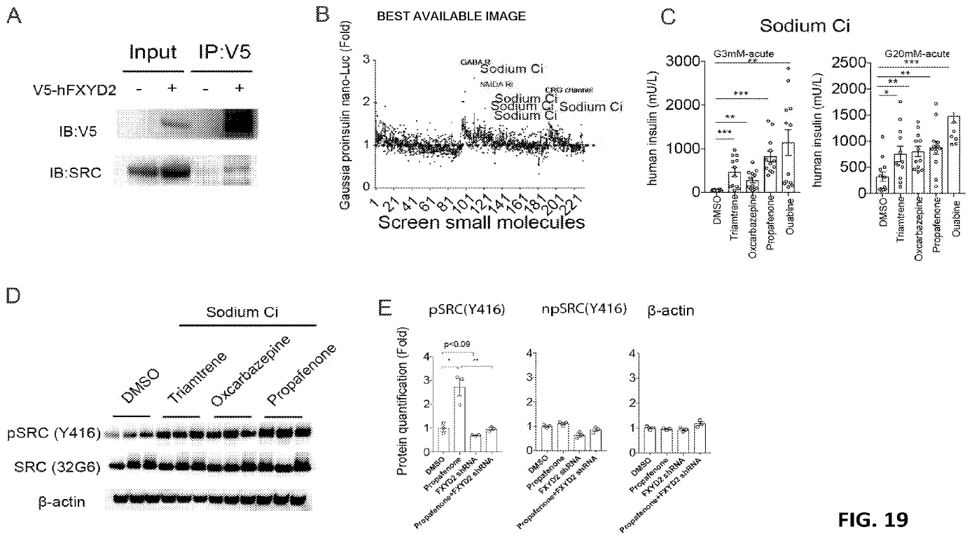

FXYD2 is known to construct the protein complex with Na+, K+ ion transporters (NKA) as a modulator of NKA activity. Our small molecules screening (via the proinsulin Nano Luc system in EndoC-BH1 cells) identified many of sodium channel modulators (Sodium Ci) enhanced insulin secretion in EndoC-BH1 cells (FIG. 19B). These sodium cis, include triamtrene, oxcarbzepine, propafenone and ouabine, showed acute stimulation of insulin secretion under low and high glucose condition in EndoC-BH1 cells (FIG. 19C). Notably, these sodium cis rapidly enhanced phosphorylation at Tyr416 in SRC (FIG. 19D). To test whether sodium cis-induced SRC activation relate to the amplification of insulin secretion, we treated SRC modulators and/or propafenone in primary human islets. We found propafenone enhanced high-glucose and KCl-induced insulin secretion, but the SRC inhibitor (dasatinib) partially suppressed insulin secretion in human islets (FIG. 18G). In contrast, SRC family activator enhanced high-glucose and KCl-induced insulin secretion, while we did not observe addictive effect in insulin secretion with propafenone treatment (FIG. 18G). In addition, we found FXYD2 knock down by FXYD2 shRNA defect sodium cis such as propafenone-induced pSRC phosphorylation in EndoC-BH1 cells (FIG. 19E). These results suggest that FXYD2/NKA/SRC axis regulates physiological human β cell insulin secretion (FIG. 19H).

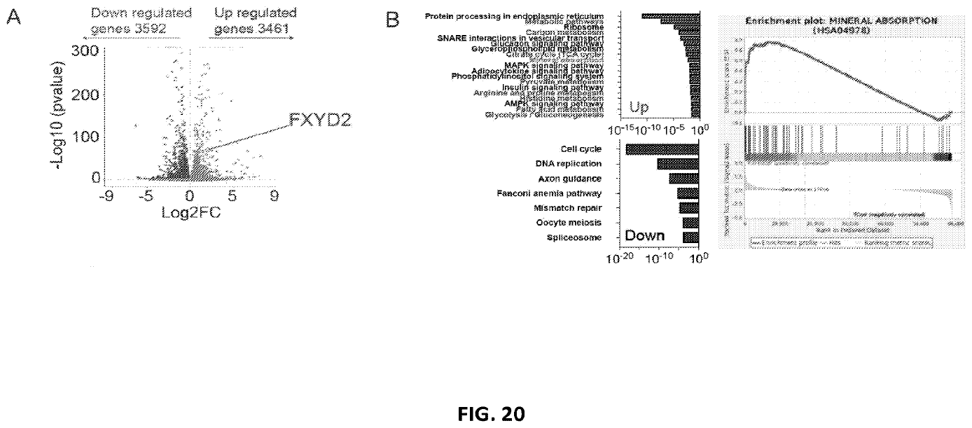

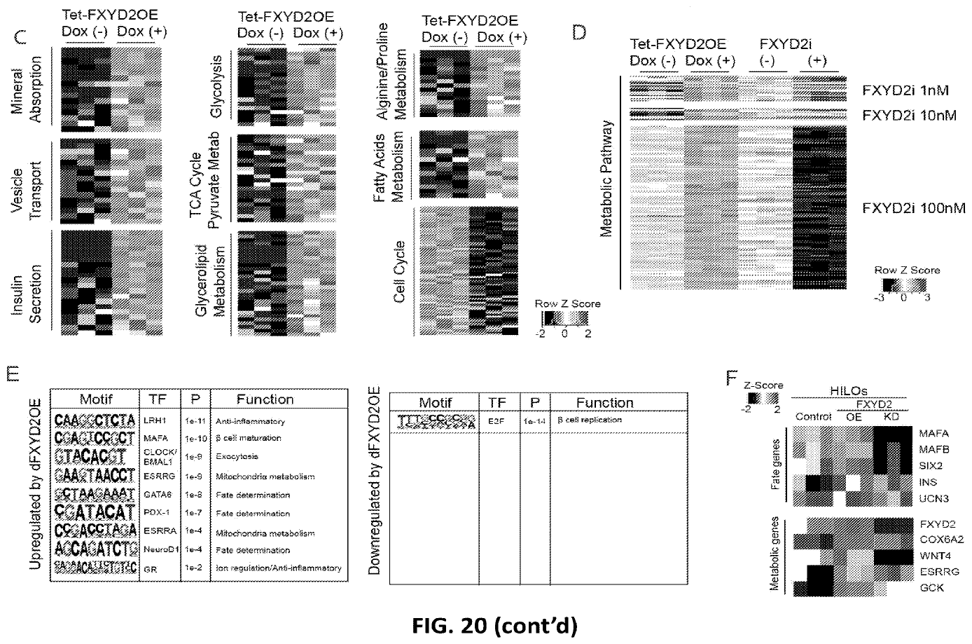

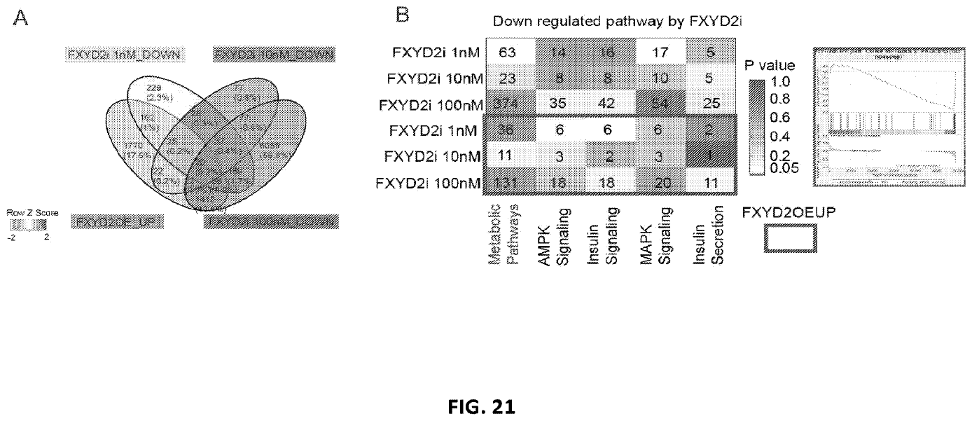

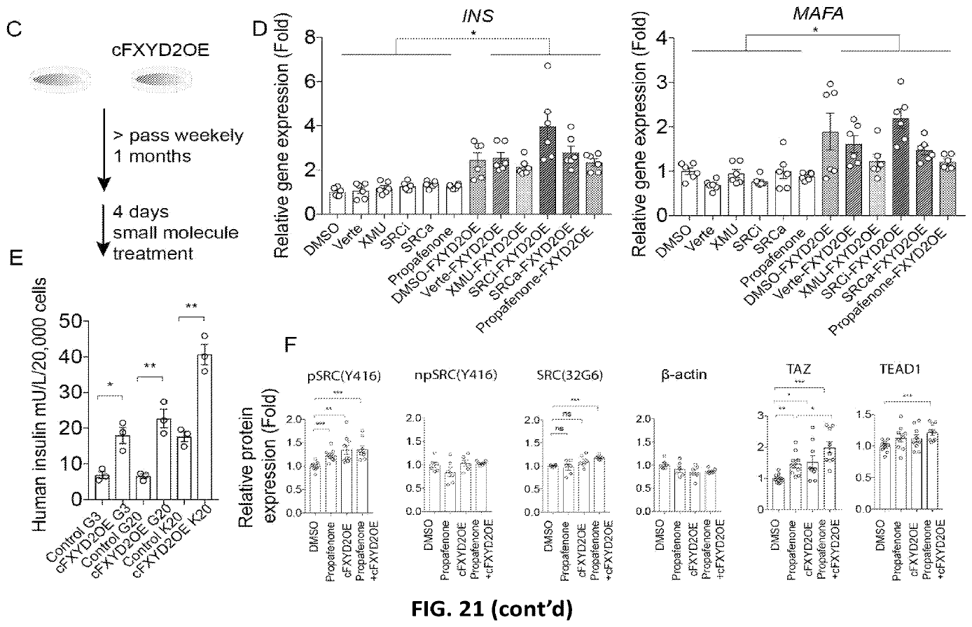

FXYD2 regulates transcriptome necessary for functional maturation in human β cells. Next, to test whether FXYD2 regulates not only β cell insulin secretion abut also β cell identity and maturity, we performed bulk RNA-seq using dFXYD2OE in EndoC-BH1 cells. We identified 3,461 genes induced by FXYD2 and 3592 genes repressed by FXYD2 (FIG. 20A). We found that FXYD2 broadly regulates the gene expression necessary for insulin secretion in EndoC-BH1 cells (FIG. 20B). We found that dFXYD2OE enhanced the gene clusters necessary for insulin secretion such as the mineral absorption pathway, vesicle transport, insulin secretion, glycolysis, TCA cycle, and other metabolic pathways (FIG. 20A-20C). In contrast, dFXYD2OE in EndoC-BH1 cells reduced the gene cluster for cell cycle progression (FIG. 20C), which correlates to postnatal functional maturation in β cells. We found that FXYD2i reciprocally reduced the gene clusters specially related to metabolic pathway and insulin secretion (FIG. 20D, 21A, 21B). These results suggest that FXYD2 expression is a novel fine-tuning system to restore the β cell transcriptomes and regulates GSIS function. We earlier identified MAFA and ESRRG as the transcriptional factors relate to postnatal maturation in islets. Our promoter motif analyses identified that the cluster of genes induced by dFXYD2OE is regulated by the transcriptional factors include MAFA and ESRRG in EndoC-BH1 cells (FIG. 20E, left). Since both MAFA and ESRRG genes were not induced by transient dox-dependent FXYD2OE in EndoC-BH1 cells, we constructed constitutive active FXYD2 expressing (cFXYD2OE) EndoC-BH1 cells (FIG. 21C). We passaged control and cFXYD2OE EndoC-BH1 cells every week for 1 months and investigated gene expression and function. We found cFXYD2OE significantly increased INS and MAFA expression in EndoC-BH1 Cells (FIG. 21D). Consistent with this result, we found low glucose (3 mM), high glucose (20 mM) and high KCl (20 mM) stimulated insulin secretion was increased in cFXYD2OE EndoC-BH1 cells (FIG. 21E). In contrast, dFXYD2OE suppressed the cluster of genes those are regulated by E2F, a known β cell replication factor in EndoC-BH1 cells (FIG. 20E, right). These results suggest that FXYD2 may regulate β cell maturity by enhancing β cell lineage gene expression and suppresses β cell replication. Enhancing β cell lineage gene expression and reducing cell cycle progression is the character of β cell maturation.

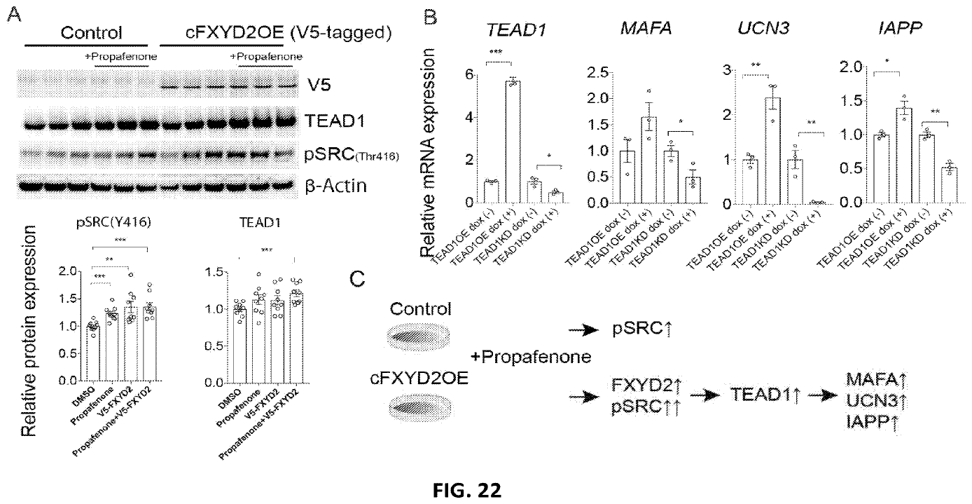

To investigate whether FXYD2 regulate human β cell maturation, we used HILOs to test the impact of dFXYD2OE and FXYD2KD in human islet organogenesis. We found dox-dependent induction of FXYD2 during the course of differentiation from day 27 to day 31 in HILOs enhanced β cell lineage gene expression such as INS, MAFA, MAFB, SIX2, UCN3 and metabolic genes relate to maturation such as COX6A2, WNT4, ESRRG, GCK (FIG. 20F). In contrast, FXYD2KD in HILOs from day 27 to day 31 reduces these genes (FIG. 20F). TEAD1, was shown as YAP/TAZ or SRC downstream molecules, which enhances maturity of β cells through direct activation of MAFA and PDX1. TEAD1 and TAZ but not YAP is expressed in matured β cells. Interestingly, FXYD2 constitutive activation with or without propafenone treatment increased pSRC (Y416) phosphorylation and TAZ and TEAD1 expression in EndoC-BH1 cells (FIG. 22A). In addition, dox-inducible TEAD1 overexpression enhanced β cell maturity related gene expression such as MAFA, UCN3 and IAPP, whereas TEAD1 knockdown reduced those of expression in EndoC-BH1 cells (FIG. 22B,C). Taken together, these results suggest that FXYD2 regulates the gene expression for β cell differentiation, maturation and function through SRC/TEAD1 signal pathway.

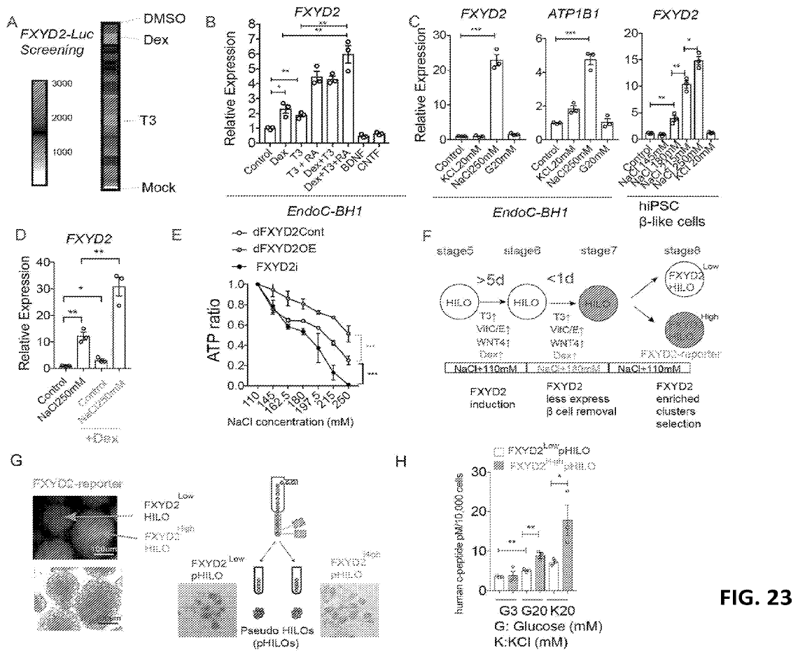

FXYD2 expression is regulated by nuclear receptor signaling and osmotic pressure. Identifying of the mechanism for how FXYDs expression is regulated in human islets may contribute to further improvement of hPSC-derived islets generation. To identify the upstream factor of FXYD2 gene expression, we have constructed an FXYD2 promoter-driven Firefly-mCherry-dual-reporter system (FXYD2-fLuc-mCherry) in EndoC-BH1 cells and performed small molecule screening by using 500 FDA-approved drug libraries (FIG. 23A). We have found that FXYD2 expression is indued by high NaCl+, dexamethasone (Dex, glucocorticoids receptor ligands) and Triiodothyronine (T3, thyroids hormone receptor ligands) in EndoC-BH1 cells (FIG. 23B). Synergistic FXYD2 upregulation by Dex and T3 was observed and these nuclear receptor dependent FXYD2 upregulation was enhanced by retinoic acids (RA, retinoic receptor ligands) (FIG. 23B). Furthermore, we found that NaCl+ osmotic pressure enhances FXYD2 expression in EndoC-BH1 cells and hiPSC derived β-like cells, respectively (FIG. 23C). NaCl+ osmotic pressure synergistically enhances the Dex/T3/RA-mediated FXYD2 expression in EndoC-BH1 cells (FIG. 23D). These results revealed that NaCl+ osmotic stress and nuclear receptor signaling synergistically regulate FXYD2 expression in human β cells at the chromatin level.

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for preparing a tissue, preferably a pancreatic islet organoid, comprising:
dispersing stem cells in a solution comprising 0.1% (w/v) to 5% (w/v) gellan gum,
dropping the solution with the dispersed stem cells to a buffer comprising Ca²⁺ to form a three-dimensional (3D) culture of stem cells, and
differentiating the stem cells in the 3D culture, thereby forming a pancreatic islet organoid comprising at least 100,000 pancreatic islet cells.

2. The method of claim 1, wherein the solution comprises 0.2% (w/v) to 2% (w/v), preferably 0.5% (w/v) to 1.5% (w/v) gellan gum.

3. The method of claim 1, wherein the solution comprises 0.2×10⁶ to 5×10⁷ stem cells, preferably 1×10⁶ to 2×10⁷ stem cells, or 1×10⁶ to 1×10⁷ stem cells.

4. The method of claim 1, wherein the stem cells are induced pluripotent stem cells (iPSC) or embryonic stem cells (ESC).

5. The method of claim 1, wherein the buffer, upon receiving the stem cells, comprises 0.05% (w/v) to 0.4% (w/v) gellan gum, preferably 0.1% (w/v) to 0.2% (w/v) gellan gum.

6. The method of claim 1, wherein the solution and the buffer each is pre-heated to about 35-39 °C prior to be in touch with the stem cells.

7. The method of claim 1, further comprising, prior to differentiation, growing the 3D culture in a growth medium.

8. The method of claim 7, wherein the growth medium comprises a Rock inhibitor, an FGF2, or the combination thereof.

9. The method of claim 1, wherein the differentiation is carried out in a differentiation medium.

10. The method of claim 9, wherein the differentiation medium comprises one or more of CHIR99021, Activin A, or KGF.

11. The method of claim 1, wherein the pancreatic islet organoid comprises at least 200,000, 300,000, 500,000, 800,000 or 1,000,000 pancreatic islet cells.

12. The method of claim 1, wherein the pancreatic islet organoid is at least 100 μm in diameter, or at least 200 μm, 300 μm, 400 μm, 500 μm, 600 μm, 700 μm, 800 μm, 900 μm, 1000 μm, 1200 μm, 1500 μm, 1800 μm, 2000 μm, 5000 μm, 8000 μm, or 10,000 μm in diameter.

13. The method of claim 1, wherein the pancreatic islet cells in the pancreatic islet organoid are capable of cell-to-cell communication.

14. The method of claim 1, wherein the pancreatic islet cells in the pancreatic islet organoid are capable of expanding into endodermal, mesodermal and/or ectodermal layers.

15. The method of claim 1, further comprising verifying the pancreatic islet organoid by detecting expression of FXYD Domain Containing Ion Transport Regulator 2 (FXYD2).

16. The method of claim 15, wherein an expression level of FXYD2 higher than a predetermined cutoff level indicates that the pancreatic islet organoid is functional.

17. A pancreatic islet organoid prepared by a method of claim 1.

18. An in vitro prepared pancreatic islet organoid, wherein the pancreatic islet organoid is at least 100 μm in diameter, or at least 200 μm, 300 μm, 400 μm, 500 μm, 600 μm, 700 μm, 800 μm, 900 μm, 1000 μm, 1200 μm, 1500 μm, 1800 μm, or 2000 μm, 5000 μm, 8000 μm, or 10,000 in diameter, comprises at least at least 200,000, 300,000, 500,000, 800,000 or 1,000,000 pancreatic islet cells, and comprises gellan gum.

19. A method of providing functional pancreatic islet cells to a patient in need thereof, comprising administering to the patient one or more pancreatic islet organoid of claim 17.

20-23. (canceled)

24. A method for improving functionality or glucose-stimulated insulin secretion (GSIS) of a pancreatic islet cell, comprising increasing the biological activity of the FXYD2 gene in the pancreatic islet cell.

25-28. (canceled)
